(19) United States
(12) Patent Application Publication
(10) Pub. No.: US 2025/0009951 A1
(43) Pub. Date: Jan. 9, 2025

(54) IMPROVED CANCER IMMUNOTHERAPY TREATMENTS

(71) Applicant: CYTOSORBENTS CORPORATION, Monmouth Junction, NJ (US)

(72) Inventors: Phillip P. CHAN, Monmouth Junction, NJ (US); Karl-Gustav RUGGEBERG, Monmouth Junction, NJ (US); Ritu TRIPATHI, Monmouth Junction, NJ (US); Ophir ORTIZ, Monmouth Junction, NJ (US); Richard R. MCKAY, Monmouth Junction, NJ (US)

(21) Appl. No.: 18/713,370

(22) PCT Filed: Nov. 29, 2022
(86) PCT No.: PCT/US2022/080587

§ 371 Date: May 24, 2024

Related U.S. Application Data
(60) Provisional application No. 63/283,697, filed on Nov. 29, 2021.

(51) Int. Cl. A61M 1/36; B01J 20/26; B01J 20/28

(52) U.S. Cl. CPC A61M 1/36; B01J 20/261; B01J 20/267; B01J 20/28073; B01J 20/28076; B01J 20/28083; B01J 20/28085

(57) ABSTRACT

Methods of treating cancer in a subject by administering a therapeutically effective amount of a porous biocompatible sorbent are disclosed. The porous biocompatible sorbents comprise a range of pore diameters between about 50 Å to about 40,000 Å and a pore volume between about 0.5 cc/g to about 5.0 cc/g dry polymer.

### CROSS REFERENCE TO RELATED APPLICATIONS

The present invention claims priority to U.S. Patent Application No. 63/283,697, filed Nov. 29, 2021, the disclosure of which is incorporated by reference in its entirety.

### TECHNICAL FIELD

The invention concerns treatments related to cancer immunotherapy.

### BACKGROUND

Cancer is the second leading cause of death behind heart disease (www.cdc.gov/nchs/fastats/leading-causes-of-death.htm), and research is abundant and ongoing to identify effective therapeutic agents for treating subjects with some form of the disease. As there is an extensive variety of cancers, it is not surprising that the pharmacopeia directed to treating or eliminating the disease is similarly extensive including pharmaceutical therapies, such as chemotherapies and immunotherapies, radiation therapy, and nuclear therapy. Cancer immunotherapy has shown tremendous promise and is based on a wide variety of biologic, drug, and cell therapies, such as CAR-T cell immunotherapy. However, these therapies are not optimal and are associated with serious adverse events. Here, we discuss the application of sorbent technologies to modulate the immune response as a primary cancer immunotherapy, as a way to improve the response to other cancer therapies, and as a way to mitigate adverse events such as Cytokine release syndrome (CRS), neurotoxicity and CAR-T cell related encephalopathy, and tumor lysis syndrome (TLS). As with any therapeutic, complications or side-effects arising from treatment can negatively impact a patient's quality of life, weaken the patient to an extent that they are vulnerable to opportunistic infection, or cause the patient to quit therapy altogether.

One example of a cancer treatment complication is cytokine release syndrome (CRS). This occurs when activated white blood cells release cytokines that then activate additional leukocytes (white blood cells) and other cells that can produce cytokines. This positive feedback loop can lead to ever-increasing levels of cytokines and may eventually result in a cytokine storm, or cytokine release syndrome (CRS). The increased concentration of cytokines observed in CRS can cause unwanted symptoms in a subject such as fever, muscle and joint pain, diarrhea, vomiting, cardiac and blood pressure changes, organ damage, organ failure, and death. In a similar phenomenon, subjects can develop immune related adverse events (irAE), Immune related events often include rash, diarrhea, autoimmune hepatitis, colitis, arthritis, pneumonitis, and endocrinopathy. Initial activation of the immune system cells can be caused by administering immunotherapies, such as monoclonal antibody therapy, agonists or antagonists to cell surface receptors or proteins, bispecific T-cell engagers, or adaptive T-cell therapies comprising T-cells expressing chimeric antigen receptors (CAR-T cell therapy). Furthermore, in some cases, increased cytokine levels can directly interfere with the cancer therapy itself.

CAR-T-cell-related encephalopathy syndrome (CRES), a neurotoxicity observed in patients receiving CAR-T therapy, can occur concurrently, during, or after CRS. Other related terms to CRES are Immune Effector Cell-Associated Neurotoxicity (ICANS) or Chimeric Antigen Receptor T Cell Therapy-Associated Toxicity (CARTOX). Also associated with increased concentration of cytokines, symptoms of severe forms of CRES can include seizures, incontinence, motor weakness, increased intracranial pressure, mental impairment, and death. In clinical trials, CRES often occurs after the onset of CRS. CRS and cytokine storm are associated with endothelial tight junction disruption, capillary leak syndrome, and a subsequent breakdown of the blood brain barrier that allows activated CAR-T lymphocytes and other leukocytes to enter the normally protected central nervous system and cause inflammation. CRES occurring after CRS typically is protracted and more severe than CRES occurring concurrently with CRS.

Cachexia is a wasting syndrome characterized by significant weight loss. It is a serious end-stage complication seen in cancer patients and a number of chronic illnesses such as AIDS (acquired immunodeficiency syndrome), chronic kidney disease, heart failure, and burn injury recovery. In addition to serious weight loss with muscle and fat wasting, cachexia often leads to significant weakness and debilitation, an inability to tolerate continued cancer treatment, and a susceptibility to life-threatening complications such as infection and sepsis. While not wanting to be bound by theory, it is believed that inflammation and increased concentrations of cytokines are at least partially responsible for cachexia manifestation and progression. Because the weight loss and debilitation associated with cachexia is not remedied by normal nutritional support treatment in many cancer patients, complications and death often results.

Another potentially fatal complication of some cancer treatments is tumor lysis syndrome (TLS). Patients are at a heightened probability of TLS when their therapy results in lysis of a large number of cancer or tumor cells and the cellular components of the lysed tumor cells are deposited in the blood stream. Often associated with blood cancers such as leukemia, TLS is characterized by high blood levels of potassium, phosphorus, uric acid, and blood urea nitrogen, and low blood levels of calcium, and symptoms can include nausea, nephropathy, acute kidney failure, seizures, cardiac arrhythmias, and death.

Complications arising from cancer and cancer treatments, such as CRS, CRES, TLS, cachexia, and other cancer-related maladies, side-effects, syndromes, and symptoms, remain a major problem when treating patients. Novel methods are needed to eliminate or lessen the severity of cancer treatment complications and improve the effectiveness of cancer therapy.

In addition to reducing the severity or incidence of complications related to cancer and cancer therapies, there is a need to improve the effectiveness of cancer therapy and to develop better treatments of cancer. Cancer cells have evolved sophisticated ways of evading identification and destruction by the host's immune system and other defenses (immune surveillance) and reducing the activity of the immune response and a broad range of immune effector leukocytes (e.g., lymphocytes, granulocytes, antigen presenting cells such as dendritic cells and macrophages, and others). For example, many cancers secrete or cause to shed from cell membrane surfaces (e.g. cancer cells, leukocytes, platelets, cells within the tumor microenvironment), soluble or extracellular vesicle-associated checkpoint molecules, which are ligands or receptors that cause immunosuppression by one of many mechanisms such as blocking leukocyte (e.g. cytotoxic T-cells, NK natural killer cells) activation, triggering inhibitory signaling pathways and immune checkpoints that decrease leukocyte activity or induce leukocyte apoptosis, or interfering with the recognition and killing cancer cells by leukocytes. These ligands often target cell-surface molecules or receptors on leukocytes that normally regulate immune cell function, such as CTLA-4 (Cytotoxic T-lymphocyte-associated protein 4), PD-1 (Programmed cell death protein 1 (PD-1), T-cell immunoglobulin mucin 3 (TIM3), B- and T-lymphocyte attenuator (BTLA), natural killer cell receptor NKG2D (natural-killer group 2, member D), lymphocyte-activation gene-3 (LAG-3), and others. Examples of these ligands include soluble or extracellular vesicle-associated PD-L1 and soluble or extracellular vesicle-associated PD-L2 ligands that activate the PD-1 receptor on cytotoxic T-cells, which downregulates T-cell activity and prevents cell-mediated killing of the tumor.

Another example includes soluble NKG2D ligands such as MHC class I chain-related protein A (sMICA) and B (sMICB), and HCMV UL-16 binding protein (sULBP2), that bind to the NKG2D receptor on natural killer cells, inducing internalization and a down-regulation of expression of the NKG2D receptor on the cell surface, and a reduced ability of these NK cells to kill tumor cells. Another example is soluble FAS ligand (sFasL) that induces apoptosis of lymphocytes. Another example is soluble CD40 ligand (sCD40L) that may promote the activity of immunosuppressive myeloid-derived suppressor cells and regulatory T-cells, resulting in immunosuppression in cancer. Yet another example is soluble CTLA-4 (sCTLA-4), an alternately spliced mRNA version of the membrane bound isoform, which can bind to CD80/CD86, downregulate T-cell responses, and cause immunosuppression. It has been observed both in animals and clinically that high levels of these ligands are associated with progressive and refractory cancers. The use of checkpoint inhibitors (e.g., drugs, small molecules, biologics, monoclonal antibodies) that prevent activation of PD-1 (e.g., Pembrolizumab and Nivolumab) or inhibit the PD-L1 ligand (e.g., Atezolizumab, Avelumab, Durvalumab) has been a promising anti-cancer approach. Similarly, inhibitors of CTLA-4 (e.g., Ipilimumab) has been another promising approach. However, these checkpoint inhibitors only target one of many different mediators that may contribute to immune evasion by the tumor and have the potential to increase autoimmunity or a cytokine release syndrome. A broader spectrum approach that can reduce many known and unknown substances in blood that contribute to tumor growth and evasion could potentially result in a more effective therapy.

Additionally, there is a need for methods to balance, prime, or condition the immunologic status of subjects before, during, or after administering cancer immune-therapeutics, including CAR-T cell immunotherapy. For example, subjects present with a wide variation in their immune response, along the full spectrum of immunosuppression to immune activation. This potentially impacts the response to cancer immunotherapies and potentially may contribute to an increased risk of adverse events such as cytokine release syndrome. A large part of this heterogeneity is caused by a broad range of immunostimulatory and immunosuppressive substances in the bloodstream that affect immune system function, such as cytokines, chemokines, soluble ligands, and others. For example, the pre-treatment of subjects to normalize their immune responses ahead of being administered the immunotherapy, may result in more consistent results with fewer adverse events.

This disclosure is directed to these and other needs.

### SUMMARY

Provided herein are methods for treating a subject with a therapeutically effective amount of a sorbent to treat dangerous or life-threatening complications of cancer therapy and immunotherapies (e.g. checkpoint inhibitors, monoclonal antibodies, bi-specific T-cell engagers, CAR-T cell immunotherapies, cancer vaccines, oncolytic viruses, cytokines, and combinations hereof), such as cytokine release syndrome, cytokine storm, tumor lysis syndrome, and CAR-T cell related encephalopathy, wherein the therapy reduces dangerous concentrations of one or more of cytokines, inflammatory mediators, toxins, electrolytes, chemicals, cellular debris, and metabolic waste products. In one embodiment, treatment with the sorbent reduces neurotoxicity and CAR-T cell related encephalopathy by reducing the entry of CAR-T cells and other activated cells into the central nervous system, by for example, reducing the damage by CRS or promoting repair of the disrupted endothelial tight junctions that can cause capillary leakage of the blood brain barrier. Methods are also provided for directly treating cancer in a subject with a therapeutically effective amount of a sorbent. In one embodiment, the sorbent can reduce inhibitors of the immune system thereby restoring immune surveillance and restoring or enabling the immune response to attack and kill cancer cells. Such a method could also be used in conjunction with other cancer therapies or immunotherapies.

Methods are also provided for treating a subject with a therapeutically effective amount of a sorbent to prime, condition, balance, or modulate the immune system before, during, or after cancer immunotherapy treatment, as a way to improve the strength of, durability of, or predictability of a response to cancer therapy, or to reduce the risk of adverse events. In one embodiment, the sorbent can be used to treat the blood and reduce excessive levels of either anti- or pro-inflammatory mediators to balance the immune response.

Methods are also provided for treating or preventing natural inflammation-driven complications of cancer such as cancer cachexia.

The sorbent disclosed in the methods of this disclosure can, in some embodiments be a porous biocompatible polymer, and in some aspects, a coated porous biocompatible polymer. In some embodiments, the porous biocompatible polymer can comprise a dispersing agent or an ion exchange polymer, and in some aspects the polymer can be a hydrogel, cellulosic, or an ion exchange polymer. The sorbent disclosed herein can have a pore diameter between about 50 Å to about 40,000 Å, and in some aspects of the present disclosure, the sorbent can comprise a heterogeneous population of polymers having pore diameters between about 50 Å to about 40,000 Å. In other aspects of the present disclosure, the sorbent can comprise a heterogeneous population of polymers having pore diameters between about 50 Å to about 10,000 Å. In still other aspects of the present disclosure, the sorbent can comprise a heterogeneous population of polymers having pore diameters between about 50 Å to about 3000 Å.

In some aspects of the present disclosure, the substance for which the sorbent is therapeutically effective is a a) cytokine or chemokine, such as a member of the interleukin, interferon, tumor growth factor, or tumor necrosis factor family, b) soluble checkpoint molecule such as the examples found in Table I, c) extracellular vesicle associated with a checkpoint molecule such as the examples found in Table I.

TABLE I
Soluble and Membrane-Associated Checkpoint Molecule Examples

| | | Approximate | | | Approximate |
|---|---|---|---|---|---|
| | Approximate | MW of | | Approximate | MW of |
| | MW of soluble | membrane- | | MW of soluble | membrane- |
| | form of | associated form | | form of | associated form |
| | checkpoint | of checkpoint | | checkpoint | of checkpoint |
| Checkpoint | molecule, if | molecule, if | Checkpoint | molecule, if | molecule, if |
| Molecule | applicable (kDa) | applicable (kDa) | Molecule | applicable (kDa) | applicable (kDa) |
| PD-L1 | 35 | 45-70 | VSIG-3 | 29 | 55-65 |
| PD-L2 | 25 | 31 | CEACAM1 | 100-130 | 160 |
| MICA | 45-60 | 62 | CD47 | 37-45 | 50 |
| MICB | 45-60 | 62 | CD200 | 41-47 | 60 |
| ULBP2 | 30-37 | 37 | Adenosine | <0.3 | — |
| FasL | 25-30 | 40 | B7-H7 | 38.4 | — |
| CD40L | 18 | 33 | PD-1 | 19 | 31 |
| Gal-3 | 25-30 | 30 | NKG2D | 43 | 67 |
| Gal-9 | 36 | 39 | FasR | 26 | 46 |
| HMGB1 | 25 | 30 | CD40 | 20-25 | 43 |
| HVEM | 30 | 45 | Tim-3 | 21 | 33 |
| MHC II | 27.8 | 45 | LIGHT | 21 | 30 |
| FGL1 | 35 | 116.3 | VISTA | 19 | 50 |
| LSECtin | 27.8 | 52.1 | BTLA | 23-33 | 53.7 |
| B7-H6 | 28 | 28-30 | Lag-3 | 60 | 70 |
| BAG6 | 118 | 180 | NKp30 | 30 | 30 |
| B7-H4 (VTCN1) | 27 | 50-80 | TIGIT | 10-24 | 150 |
| B7-H3 | 31 | 39 | CTLA-4 | 15.4 | 33-37 |
| PVR (CD155) | 35.3 | 70 | CEACAM1 | 100-130 | 160 |
| PVRL2 (CD112) | 36.3 | 70 | SIRP alpha | 44-63 | 75 |
| Nectin-3 (CD113) | 38.7 | 83 | CD200R | 50-70 | 70-115 |
| Nectin-4 | 43 | 66 | ADORA1 | — | 37 |
| B7-1 (CD80) | 25-45 | 33 | ADORA2a | — | 36 |
| B7-2 (CD86) | 26-57 | 70 | TMIGD2 | — | 21-34 |
| | | | ICOS | 20-34 | 55-60 |

Some embodiments of the methods recited herein are used in treating subjects in need of immunotherapy and further comprise administering an immunotherapy. In some embodiments, administration of the immunotherapy is subsequent to the administration of the sorbent. This administration of a sorbent and an immunotherapy, in some aspects of the present disclosure, elicit harmonized immune responses in the subjects.

### BRIEF DESCRIPTION OF THE DRAWINGS

FIG. 1 presents data from a benchtop evaluation showing the average percent removal of sPD-L1 using CytoSorb versus Control from bovine whole blood. The data represents mean S.D.; each group was evaluated three times (n=3). CytoSorb 60 and 120 indicate the volume of CytoSorb used (60 mL and 120 mL, respectively). Control 60 and 120 are the controls (sham, no CytoSorb) that were evaluated at the same time as the treatment (CytoSorb) groups.

FIG. 2A presents data directed to average percent remaining of sCTLA-4, sTIM-3, sLAG-3 and sPD-L1 in bovine plasma in control and CytoSorb groups. The data represents mean±S.D. (n=3), *p<0.05. FIG. 2B presents data directed to average percent remaining of sPD-L2, sPD-1, sICOS and sVISTA in bovine plasma in control and CytoSorb groups. The data represents mean±S.D. (n=3), *p<0.05, n=2 for sPD-1.

### DETAILED DESCRIPTION OF ILLUSTRATIVE EMBODIMENTS

The instant invention relates generally to cancer treatment utilizing a porous biocompatible polymer sorbent. In a first scenario, sorbent used to treat cancer by removing factors that inhibit the immune response to the cancer cells. In a second scenario, sorbent modulates the immune system before, during or after treatment of a subject with cancer therapy or immunotherapy. In a third scenario, sorbent lessens inflammation-driven complications of cancer (such as cachexia). In a fourth scenario, sorbent improves cancer therapy or immunotherapy effectiveness.

As required, detailed embodiments of the present invention are disclosed herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

The present invention may be understood more readily by reference to the following detailed description taken in connection with the accompanying figures and examples, which form a part of this disclosure. It is to be understood that this invention is not limited to the specific materials, devices, methods, applications, conditions or parameters described and/or shown herein, and that the terminology used herein is for the purpose of describing particular embodiments by way of example only and is not intended to be limiting of the claimed invention. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention, which are, for clarity, described herein in the context of separate embodiments, may also be provided in combination in a single embodiment. Conversely, various features of the invention that are, for brevity, described in the context of a single embodiment, may also be provided separately or in any subcombination. Further reference to values stated in ranges includes each and every value and combination of values within that range.

### Definitions

The following definitions are used herein. Unless otherwise specified, terms have their commonly used meaning in the art.

Throughout the description and claims of this specification, the words “comprise” and “contain” and variations of the words, for example “comprising” and “comprises”, mean “including but not limited to”, and are not intended to (and do not) exclude other components.

The term “biocompatible” is defined to mean the sorbent is capable of coming in contact with physiologic fluids, living tissues, or organisms, without producing unacceptable clinical changes during the time that the sorbent is in contact with the physiologic fluids, living tissues, or organisms.

As used herein, the term “treat”, “treating”, or “treatment” of any disease, condition, syndrome or disorder refers, in one embodiment, to ameliorating the disease, condition, syndrome or disorder (i.e., slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome, or disorder either physically (e.g., stabilization of a discernible symptom), physiologically, (e.g., stabilization of a physical parameter), or both. In yet another embodiment, “treat”, “treating”, or “treatment” refers to preventing or delaying the onset or development or progression of the disease, condition, syndrome, or disorder.

The term “hemocompatible” is defined as a condition whereby a biocompatible material when placed in contact with whole blood or blood plasma results in clinically acceptable physiologic changes.

As used herein, the term “physiologic fluids” are liquids that originate from the body and can include, but are not limited to, nasopharyngeal, oral, esophageal, gastric, pancreatic, hepatic, pleural, pericardial, peritoneal, intestinal, prostatic, seminal, vaginal secretions, as well as tears, saliva, lung, or bronchial secretions, mucus, bile, blood, lymph, plasma, serum, synovial fluid, cerebrospinal fluid, urine, and interstitial, intracellular, and extracellular fluid, such as fluid that exudes from burns, wounds, or cancerous tumors.

As used herein, the term “sorbent” includes adsorbents and absorbents.

For purposes of this invention, the term “sorb” is defined as “taking up and binding by absorption and/or adsorption”.

As used herein, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. Also, the term “about” when used in reference to numerical ranges, cutoffs, or specific values is used to indicate that the recited values may vary by up to as much as 10% from the listed value. Use of the antecedent “about” is understood to be inclusive of the particular value recited. All ranges are inclusive and combinable.

As used herein, the phrase “therapeutically effective dose” refers to an amount of an administered sorbent or other therapeutic that is effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the treatment of cancer, as determined by any means suitable in the art.

The term “subject” as used herein is intended to mean any animal, in particular, mammals, and any type of mammal can be treated using the disclosed methods. Thus, the methods are applicable to human and nonhuman animals, although preferably used with mice and humans, and most preferably with humans. “Subject” and “patient” are used interchangeably throughout this disclosure.

The phrase “monomer residue” refers to the portion of a monomer that is incorporated into a polymer when the monomer is polymerized. For example, when R-x is reacted with R′-y to produce R—R′ with x and y being freed during the reaction, R and R′ are monomer residues.

For the purposes of this invention, the term “perfusion” is defined as passing a physiologic fluid, once through or by way of a suitable extracorporeal circuit, through a device containing the porous polymeric adsorbent to remove toxic molecules from the fluid.

The term “hemoperfusion” is a special case of perfusion where the physiologic fluid is blood.

The term “dispersant” or “dispersing agent” is defined as a substance that imparts a stabilizing effect upon a finely divided array of immiscible liquid droplets suspended in a fluidizing medium.

The term “heparin mimicking polymer” refers to any polymer that possesses the same anticoagulant and/or antithrombogenic properties as heparin. In some embodiments, the sorbent can act as a heparin mimicking polymer by having functional groups selected from —SO₃H, —COOH and —OH (or salts thereof) on the sorbent surface. These functional groups may be attached to the sorbent polymer by means known to those skilled in the art. See, for example, Ran, et al. Macromol. Biosci. 2012, 12(1), 116-25. Suitable salts include sodium and potassium salts such as —SO₃⁻M⁺, —COO⁻M⁺, and —O⁻M⁺ where M is Na or K.

The term “macroreticular synthesis” is defined as a polymerization of monomers into polymer in the presence of an inert precipitant which forces the growing polymer molecules out of the monomer liquid at a certain molecular size dictated by the phase equilibria to give solid nanosized microgel particles of spherical or almost spherical symmetry packed together to give a bead with physical pores of an open cell structure [U.S. Pat. No. 4,297,220, Meitzner and Oline, Oct. 27, 1981; R. L. Albright, Reactive Polymers, 4, 155-174(1986)].

The term “hypercrosslinked” describes a polymer in which the single repeating unit has a connectivity of more than two. Hypercrosslinked polymers are prepared by crosslinking swollen, or dissolved, polymer chains with a large number of rigid bridging spacers, rather than copolymerization of monomers. Crosslinking agents may include bis(chloromethyl) derivatives of aromatic hydrocarbons, methylal, monochlorodimethyl ether, and other bifunctional compounds that react with the polymer in the presence of Friedel-Crafts catalysts [Tsyurupa, M. P., Z. K. Blinnikova, N. A. Proskurina, A. V. Pastukhov, L. A. Pavlova, and V. A. Davankov. “Hypercrosslinked Polystyrene: The First Nanoporous Polymeric Material.” Nanotechnologies in Russia 4 (2009): 665-75.]

### Cancer and Use of Sorbents of the Invention in the Treatment Thereof

Cancer therapies and immunotherapies are administered by convention techniques that are well known in the art.

The biocompatible polymer sorbents, in some embodiments, act to remove undesirable compounds by sorbing these unwanted compounds within pores of the sorbent and thereby treating cancer.

PD-1 is a protein is found on immune cells called T cells. PD-1 typically acts as an “off switch” preventing T cells from attacking other cells in the body. PD-1 can attach to PD-L1, a protein found on some normal (and cancer) cells. This interaction signals the T cell to not attack the cell. Certain cancer cells have significant amounts of PD-L1, which allows the cancer cells to evade the immune system. The list of cancer types expressing PD-L1 is large and includes non-small cell lung cancer, natural killer/T-cell lymphoma, diffuse large B-cell lymphoma, Hodgkin Lymphoma, multiple myeloma, oral squamous cell carcinoma, breast, ovary, urothelial, and colon carcinomas, and malignant melanoma. High levels of PD-L1 frequently correlate with poor clinical outcomes.

Therapies that target either PD-1 or PD-L1 can stop the proteins from attaching to one another and help prevent cancer cells from evading the immune system. The present disclosure provides methods of modulating cytokine concentration in a subject, which may benefit those either receiving or preparing to receive an immunotherapy for the treatment of cancer. Modulation of cytokine concentrations is a significant advance in cancer therapeutics as some cytokines are known to inhibit immune responses against tumors. Thus, one embodiment of the present disclosure is a method of enhancing responsiveness to immunotherapy in a subject comprising administering to the subject a therapeutically effective amount of a sorbent for an immunosuppressive cytokine. In certain embodiments, the methods of the invention target PD-L1 by using the sorbent to sorb PD-L1.

PD-L2 is a second ligand for PD-1 that can inhibit T-cell interaction with cells.

sPD-1, a soluble form of PD-1, has been associated with poor outcome in certain cancers.

sMICA is a soluble ligand for the immune receptor natural killer receptor (NKG2D). sMICB and sULBP2 are other soluble ligands for NKG2D.

Fas receptors are death receptors found on the surface of cells and can lead to programmed cell death. Fas ligand (FasL) is known to allow Fas to avoid immune surveillance.

sCTLA-4 is a soluble form of cytotoxic T lymphocyte-associated antigen-4 (sCTLA-4).

sCD40L is a soluble ligand that can trigger release of certain inflammatory mediators.

Gal-9 is a soluble ligand for T cell immunoglobulin and mucin domain-containing protein 3 (TIM3). sHMGB1 is another soluble ligand for TIM-3.

sHVEM is a soluble ligand for B- and T-lymphocyte attenuator (BTLA).

LIGHT is a tumor necrosis factor superfamily ligand that binds HVEM.

VSIG-3 is a ligand for V-domain Ig suppressor of T cell activation (VISTA)

FGL1 is a soluble ligand for lymphocyte-activation gene 3 (LAG-3). MHC class II, Gal-3 and LSECtin are other soluble ligands for LAG-3.

sB7-H6 is a soluble ligand for natural killer cell activation receptor NKp30. sBAG6 and Gal-3 are other soluble ligands for NKp30.

sB7-H4 (sVCTN1) and sB7-H3 are soluble immune checkpoint molecules.

sPVR (sCD155) is a soluble ligand for T cell immunoreceptor with immunoglobulin and ITIM domain (TIGIT). sPVRL2 (sCD112), sNectin-3, and sNectin-4 are other soluble ligands for TIGIT.

sB7-1 is a soluble ligand for CTLA-4. sB7-2 is another soluble ligand for CTLA-4.

sB7H7 is a soluble ligand for the receptor transmembrane and immunoglobulin domain containing 2 (TMIGD2).

Adenosine is a soluble ligand for the adenosine A1 receptor (ADORA1). Adenosine is also a ligand for the adenosine A2a receptor (ADORA2a).

sCEACAM1 is the soluble form of carcinoembryonic antigen-related cell-adhesion molecule 1 (CEACAM1).

sCD-47 is a soluble ligand for the signal regulatory protein alpha (SIRP alpha).

sCD200 is a soluble ligand for the CD200 receptor (CD200R).

Inducible T-cell Costimulator (ICOS) is a CD28-superfamily costimulatory molecule that is expressed on activated T cells. It is thought to be important for T-helper cells in particular. It plays an important role in cell-cell signaling, immune responses and regulation of cell proliferation.

A checkpoint inhibitor is a drug that can block certain proteins that are made by immune system cells. Existence of these proteins can block T-cells and other immune system cells from attacking cancer cells.

CAR T-cell therapy modifies a portion of a subject's T cells so to improve their effectiveness in attaching cancer cells.

Monoclonal antibodies are a type of protein (typically made in a laboratory) that can bind to cancer cells and can be used in the treatment of cancer.

Bi-specific T-cell engagers are a class of bispecific monoclonal antibodies that are useful in cancer treatment by linking T-cells to cancer cells.

A cancer vaccine is a vaccine that can treat an existing cancer or prevent a cancer from forming.

Oncolytic viruses are viruses that preferentially lyse cancer cells but not normal cells.

Immunotherapy using the polymer sorbents of the invention as contemplated herein refers to therapeutic approaches to provoke an immune response against a tumor or cancer cell expressing an antigen. Immunotherapy, once administered, activates white blood cells that release cytokines, both proinflammatory and anti-inflammatory. Because some cytokines are immunosuppressive and can dampen or even eliminate immune responses, another embodiment of the present disclosure is a method to enhance an immune response in a subject comprising administering to the subject a therapeutically effective amount of a sorbent for a cytokine.

The immune systems of mammals, and particularly in humans, recognize some antigens as foreign and respond with adaptive (antibody-mediated responses) and innate responses (cytokine-mediated responses), and although cancer and tumor cells are derived from non-foreign cells, they do express antigens (“tumor antigens”) that can be recognized as foreign and elicit an immune response. Some cancer and tumor cells may express certain immunosuppressive cytokines that reduce the immune system's ability to recognize tumor antigens and respond appropriately. For example, tumor growth factor-beta (TGF-beta) is known to blunt the immune system's ability to recognize tumor antigens and its overexpression is correlated with tumor progression. Administration of a sorbent to reduce the concentration and/or availability of TGF-beta, may enhance the immune system's recognition of the tumor antigen and the activation of effector cells to attack the tumor or cancerous cell expressing the tumor antigen. For these reasons, one embodiment contemplated herein is a method for enhancing an immune response in a subject comprising administering to the subject a therapeutically effective amount of a sorbent for a cytokine.

The sorbents may be used to treat cancer directly as described herein. Alternately, the sorbents may be utilized with one or more treatments selected from anticancer agents, anticancer treatments, and immunotherapeutic agents.

Without being bound by theory, it is believed that the sorbent described herein will sorb a sufficient amount of immunosuppressive cytokines such that the presence of tumor antigens will not be masked, and the immune system will generate a response against the tumor or cancerous cell expressing the antigen.

While TGF-beta, for example, and other cytokines possess immunosuppressive potential that may blunt the immune system's ability to attack cancerous cells and/or tumors, this ability may be restored to the immune system upon removal of the offending cytokine(s). Additionally, in some cancer cases, traditional therapies may not be warranted or desired, or may be contraindicated, due to a patient's medical history, status, or personal preferences; therefore, the patient may rely solely on his/her own immune response to combat the cancer. Administering the sorbent to the patient lowers the concentration of immunosuppressive cytokines resulting in an enhanced immune response (compared to a response inhibited by the immunosuppressive cytokine) that can proceed to clear the cancerous cells.

As noted supra, there are also numerous examples of cytokine-mediated syndromes, disorders, or other maladies that can arise as complications from immunotherapy. For this reason, one embodiment of the present disclosure is directed to methods of treating or preventing a cytokine-mediated malady in a subject comprising administering to the subject a therapeutically effective amount of a sorbent for a cytokine. In one aspect of this embodiment, the malady is cachexia, CRS, CRES, TLS, or a combination thereof. In other embodiments, the malady can be systemic inflammation

CRS and CRES are known to occur simultaneously in some subjects receiving cancer therapy, and because cachexia is such a prominent side effect of cancer treatments, one would expect cachexia to be comorbid with any one of the listed syndromes or other cancer-related side effects or complications. Thus, one aspect of the present disclosures is a method for treating or preventing CRS and CRES in a subject comprising administering to the subject a therapeutically effective amount of a sorbent for a cytokine. Another aspect of the present disclosure is a method of treating or preventing CRS and TLS comprising administering to the subject a therapeutically effective amount of a sorbent for a cytokine. Another aspect of the present disclosure is a method for treating or preventing CRS and cachexia comprising administering to the subject a therapeutically effective amount of a sorbent for a cytokine. In yet another aspect of the present disclosure, methods are provided for the treatment or prevention of CRES and TLS comprising administering to the subject a therapeutically effective amount of a sorbent for a cytokine. Another aspect provides a method of treating or preventing CRES and cachexia comprising administering to the subject a therapeutically effective amount of a sorbent for a cytokine. Another aspect of the present disclosure provides a method for treating or preventing TLS and cachexia.

Yet other aspects of the invention use the sorbents described herein as a pre-treatment to immunomodulate patients that are planning to undergo cancer immunotherapy to homogenize the patient population, reduce the incidence of adverse outcomes (e.g., lower CRS, CRES, TLS, and cachexia), and/or improve clinical outcomes (e.g., improved tumor antigen response rates, increased survivability, and decreased recurrence rates). Some patients are ineligible for immunotherapy treatment because their T-cells will not multiply outside of the body and provide successful treatment when returned back within the patient's body. The instant methods promote overcoming this issue and allowing more patients to be eligible for treatment.

Another embodiment of the present invention provides administering to a subject a therapeutically effective amount of a sorbent to reduce the concentration of a cytokine, wherein the reduced concentration of the cytokine conditions the subject for a subsequent administration of Chimeric Antigen Receptor T-Cell Therapy (CAR-T). Subject conditioning can increase the probability that subsequent immunotherapies such as CAR-T will be effective. The conditioning of subjects can also be used to harmonize a group of subjects such that subsequent treatments for the group can occur at approximately the same time. Implementing this harmonization of subjects is ideal in research settings, but also in settings that may not be equipped to administer expensive cancer therapies without sufficient preparatory time. For example, clinics serving underprivileged patients or underserved locations may need to purchase multiple units of a therapy to take advantage of bulk-pricing or mobile clinics may only serve a particular location on an interval basis. In such circumstances, harmonizing patients allows for the efficient distribution of treatment and better outcomes.

The invention also concerns treatment of cancer cachexia (also called cancer anorexia cachexia). Although cachexia is a common occurrence in cancer, and at times a remarkably visible indicator of the state of one's illness, more concern and attention is directed to treating the cancer rather than this complication. However, the deprivation of nutrition, characteristic of cachexia, causes many patients to be in a weakened state and unable to receive advanced treatments, and for those whose cancer is intractable, palliative care is the last resort. Some embodiments of the present disclosure are methods for treating or preventing cachexia in a subject comprising administering a therapeutically effective amount of a sorbent for a cytokine. As with other methods presented herein, the sorbent may be administered prior to, concomitant with, or subsequent to, administration of an anti-cancer therapy. By decreasing the concentration of cytokines, cachexia can be prevented, eliminated, or the symptoms reduced to a level which will allow patients to receive nourishment and better combat the underlying disease.

One aspect of the methods disclosed herein towards treating or preventing a cytokine-mediated malady is the reduction of cytokine concentration. In some aspects of the disclosed embodiments the reduction in the concentration of a single class, or even a specific, cytokine can be 5%, 10%, 15%, 20%, 25%, 30%, 35%, 40%, 45%, 50%, 55% 60%, 65%, 70%, 75%, 80%, 85%, 90%, 91%, 92%, 93%, 94%, 95%, 96%, 97%, 98%, 99%, or even 100%. A 100% reduction in the concentration of a cytokine describes a situation in which the cytokine(s) is/are not detectable by any method used to detect cytokine levels in a subject.

The cytokine(s) being bound and effectively neutralized can be a single species of cytokine, a particular family of cytokines, a panel of cytokines, or even a random set of cytokines. Cytokines that may be sorbed using the methods and sorbents described herein can be immunosuppressive, pro-inflammatory, or having cancer-promoting qualities. Examples of cytokines contemplated in this disclosure are interleukins (IL), members of tumor necrosis family (TNF), interferons (IFN), and transforming growth factors (TGF). In some aspects, the cytokines of interest are IL-2, IL-4, IL-6, IL-10, and IL-12. In some aspects, the cytokines of interest include IFN-γ. In some aspects, the cytokines of interest include TGF-β.

Another embodiment of the present invention provides methods for treating or preventing tumor lysis syndrome in a subject comprising administering to the subject a therapeutically effective amount of a sorbent for a cytokine. TLS occurs when treatment causes the lysis of a large number of tumor cells, thereby releasing the cellular contents of the lysed cells. Not only are the cellular contents released into the extracellular milieu or blood stream, but cytokines are also released. The sudden increase in cytokines can elicit a systemic inflammation response that can lead to organ failure and death. Because a sorbent effective for a cytokine can reduce the amount of cytokines, the methods described can reduce the systemic inflammation associated with TLS by sorbing the released cytokines.

Additionally, the administered therapeutically effective amount of a sorbent may also sorb cellular debris. As used herein, “cellular debris” refers to the organic waste that remains after a cell dies, and the immune system is often involved in clearing the debris. In some instances, such as TLS, the amount of cellular debris released by a dying tumor is sufficient to adversely impact a subject's health. By sorbing at least some portion of the cellular debris, the body is able to clear the debris more efficiently than relying on the immune system alone. Such application of the present invention may prove helpful in immunocompromised individuals whose immune system cannot effectively clear the debris.

In addition, the invention may be used as a primary therapy for cancer by removing soluble or extracellular vesicle-associated inhibitors of the immune response. In many cases, these soluble inhibitors are classified as immune system checkpoint molecules. Thus, one embodiment of the present disclosure is a method for treating cancer in a subject comprising administering to the subject a therapeutically effective amount of a sorbent for an immune system checkpoint molecule. Checkpoint molecules include, but are not limited to, programmed cell death protein 1, also known as PD-1, its ligands PD-L1 and PD-L2, and CTLA-4. For example, the instant sorbents can remove PD-1, from the bodily fluid of the patient, and an effective immune response may be mounted against a cancer or tumor cell.

In addition to directly binding immune system checkpoint molecules, the sorbent may be administered along with a checkpoint inhibitor. This may be desirable in cases where the checkpoint inhibitor is toxic at elevated levels or is toxic at normal levels in some patients. Additionally, while checkpoint inhibitors tend to be specific for a particular checkpoint molecule, the sorbents are able to sorb any of the checkpoint molecules, especially any at an abnormally high concentration.

Some embodiments of the methods described herein may involve treating blood or other bodily fluids with at least one of the disclosed porous biocompatible polymers. For example, blood from a patient may be processed external to the patient in a manner similar to dialysis. After the blood is removed from a patient and before its return to the patient, the blood is contacted by the disclosed porous biocompatible polymer. In some embodiments, blood is taken from an appropriate artery, contacted with sorbent and then returned to within the patient through an appropriate vein. Methods of establishing a circuit to remove and return blood to the patient's body are well known by those skilled in the art. In some aspects, the porous biocompatible polymers will sorb circulating cytokines or other biomolecules that may impede cancer therapy, effectively removing them from circulating blood. Thus, once the blood is returned to the patient, the reduced concentration of the inhibiting cytokine or biomolecule will be less likely to suppress, counteract, or otherwise inhibit the immunotherapy.

In some embodiments of the present disclosure, the sorbent may be administered as an adjuvant therapy to reduce the incidence of recurrence of cancer in a patient. For example, after surgical excision of a tumor, the sorbent may be administered to reduce the concentration of immunosuppressive cytokines or other cancer-promoting factors, thus allowing the immune system to surveil for any circulating cancer cells or early tumor development.

In some embodiments, the sorbents may be contained within a cartridge adapted to allow blood to flow into the cartridge and then exit the cartridge and then be returned to the patient's body. Although cartridges are typically utilized, other containers that allow blood to contact the sorbent and then be returned to the patient may also be utilized. One embodiment of the present disclosure relates to kits comprising a cartridge comprising a sorbent for use in cancer immunotherapy treatment. The kits may contain any of the polymers described herein. In some aspects, the kit comprises instructions for use of the cartridge, and in some aspects the instructions are contained on a label attached to the cartridge.

Alternatively, sorbents can be administered directly to the patient. For example, a therapeutically effective amount of sorbent can be administered intravenously, intramuscularly, intratumorally, intradermally, intraperitoneally, subcutaneously, topically, orally, nasally, via a feeding tube, or rectally. In some instances, the sorbent may be administered directly to a tumor or to the tissues surrounding the tumor, which can result in less of a systemic effect and may be especially beneficial when anti-cancer treatments are also administered intratumorally.

In some aspects of the presently described methods, the sorbent is administered orally. This is a particularly effective means of systemically delivering the sorbent, and may be especially beneficial for patients experiencing CRS, CRES, TLS, or cachexia. Oral administration does not involve invasive or lengthy procedures, which tend to be expensive.

Additionally, oral administration allows for relatively easy and frequent (e.g., daily) administration compared to other means of administering therapeutics.

Sorbents according to the instant disclosure that may be suitable for use in treating cancer or any of the other methods/uses described herein are biocompatible polymer sorbents having the following characteristics:
a range of pore diameters between about 50 Å to about 3000 Å, alternatively from about 50 Å to about 10,000 Å, alternatively from about 50 Å to about 40,000 Å, alternatively from about 50 Å to about 2000 Å, alternatively from about 100 Å to about 1500 Å;
alternatively from about 1500 Å to about 6000 Å; alternatively from about 1500 Å to about 15,000 Å; alternatively from about 5000 Å to about 20,000 Å; alternatively from about 4000 Å to about 40,000 Å; and
a pore volume between about 0.5 cc/g to about 3.0 cc/g dry polymer, alternatively a pore volume between about 0.5 cc/g to about 4.0 cc/g dry polymer, alternatively a pore volume between about 0.5 cc/g to about 5.0 cc/g dry polymer.

In certain embodiments, the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

When used to treat cancer or in related methods discussed herein, it is contemplated that the sorbent may be placed in close proximity to the cancer. In certain embodiments, the sorbent may be placed inside the tumor. It is also contemplated that the sorbent treats cancer by selectively sorbing protein secreted by the cancer that have an effect on the immune system.

Various non-limiting scenarios illustrate the treatments of the instant invention:
Scenario 1. Sorbent used to treat cancer by removing factors that inhibit the immune response to the cancer cells.
Scenario 2. Sorbent modulates the immune system before, during or after treatment of a subject with cancer therapy or immunotherapy.
Scenario 3. Sorbent lessens inflammation-driven complications of cancer (such as cachexia).
Scenario 4. Sorbent improves cancer therapy or immunotherapy effectiveness.

Certain embodiments of the invention are directed to methods of treating cancer in a subject in need thereof using the sorbents of the disclosure. These methods may: (a) reduce the concentration of one or more of cytokines, inflammatory mediators, toxins, electrolytes, chemicals, cellular debris, and metabolic waste products in the patient relative to an untreated subject; (b) reduce the entry of CAR-T cells and other activated cells into the central nervous system relative to an untreated subject; (c) reduce inhibition of the immune system; and/or (d) reduces the damage by cytokine release syndrome (CRS) relative to an untreated subject.

In one embodiment of the invention, the method of treating cancer includes administering a porous biocompatible polymer sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

In another embodiment of the invention, the method of treating cancer includes administering a porous biocompatible polymer sorbent to the subject, wherein the polymer sorbent comprises a range of pore diameters between about 50 Å to about 10,000 Å and a pore volume between about 0.5 cc/g to about 4.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

In another embodiment of the invention, the method of treating cancer includes administering a porous biocompatible polymer sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

Another embodiment of the methods of treating cancer includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer. In that embodiment, the sorbent may be extracorporeal. The physiologic fluid may be blood.

Another embodiment of the methods of treating cancer includes contacting a physiologic fluid of the subject with a porous biocompatible polymer sorbent comprising a range of pore diameters between about 50 Å to about 10,000 Å and a pore volume between about 0.5 cc/g to about 4.0 cc/g dry polymer. In that embodiment, the sorbent may be extracorporeal. The physiologic fluid may be blood.

Another embodiment of the methods of treating cancer includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer. In that embodiment, the sorbent may be extracorporeal. The physiologic fluid may be blood.

Another embodiment of the methods of treating cancer includes providing to the subject a porous biocompatible polymer sorbent comprising a range of pore diameters between about 50 Å to about 3000 Å and a pore volume between about 0.5 cc/g to about 3.0 cc/g dry polymer.

Another embodiment of the methods of treating cancer includes providing to the subject a porous biocompatible polymer sorbent comprising a range of pore diameters between about 50 Å to about 10,000 Å and a pore volume between about 0.5 cc/g to about 4.0 cc/g dry polymer.

Another embodiment of the methods of treating cancer includes providing to the subject a porous biocompatible polymer sorbent comprising a range of pore diameters between about 50 Å to about 40,000 Å and a pore volume between about 0.5 cc/g to about 5.0 cc/g dry polymer.

In certain embodiments of the methods of treating cancer, the sorbent may be used in combination with an anticancer agent, anticancer treatment, and/or immunotherapeutic agent.

The sorbent administered prior to, or at the same time as said anticancer agent, anticancer treatment, and/or immunotherapeutic agent. The anticancer agent, anticancer treatment, and/or immunotherapeutic agent may be one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

When used in the methods of treating cancer, the sorbent may result in removal of one or more of a) cytokine or chemokine, b) soluble checkpoint molecule, and c) extracellular vesicle associated with a checkpoint molecule from bodily fluid of the subject. The soluble or extracellular vesicle-associated checkpoint molecule may be one or more of the examples found in Table I. The chemokine may include one or more of a member of the interleukin, interferon, tumor growth factor, or tumor necrosis factor family. In a specific embodiment, the soluble ligand is PD-L1.

Another embodiment of the invention is a porous biocompatible polymer sorbent comprising a range of pore diameters between about 50 Å to about 3000 Å and a pore volume between about 0.5 cc/g to about 3.0 cc/g dry polymer for treating cancer. Another embodiment is a porous biocompatible polymer sorbent comprising a range of pore diameters between about 50 Å to about 3000 Å and a pore volume between about 0.5 cc/g to about 3.0 cc/g dry polymer for use in the manufacture of a medicament for treating cancer.

Another embodiment of the invention is a porous biocompatible polymer sorbent comprising a range of pore diameters between about 50 Å to about 10,000 Å and a pore volume between about 0.5 cc/g to about 4.0 cc/g dry polymer for treating cancer. Another embodiment is a porous biocompatible polymer sorbent comprising a range of pore diameters between about 50 Å to about 10,000 Å and a pore volume between about 0.5 cc/g to about 4.0 cc/g dry polymer for use in the manufacture of a medicament for treating cancer.

Another embodiment of the invention is a porous biocompatible polymer sorbent comprising a range of pore diameters between about 50 Å to about 40,000 Å and a pore volume between about 0.5 cc/g to about 5.0 cc/g dry polymer for treating cancer. Another embodiment is a porous biocompatible polymer sorbent comprising a range of pore diameters between about 50 Å to about 40,000 Å and a pore volume between about 0.5 cc/g to about 5.0 cc/g dry polymer for use in the manufacture of a medicament for treating cancer.

Other embodiments of the invention are methods of reducing inflammation related complications of (a) cancer or (b) cancer therapy and/or immunotherapy in a subject in need thereof using the sorbents of the disclosure. In one embodiment, the methods reduce inflammation related complications of cancer. In another embodiment, the methods reduce inflammation related to complications of cancer therapy. In another embodiment, the methods reduce inflammation related complications of immunotherapy.

In one embodiment, the method of reducing inflammation related complications of (a) cancer or (b) cancer therapy or immunotherapy in a subject in need thereof includes administering a porous biocompatible polymer sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

In another embodiment, the method of reducing inflammation related complications of (a) cancer or (b) cancer therapy or immunotherapy in a subject in need thereof includes administering a porous biocompatible polymer sorbent to the subject, wherein the polymer sorbent comprises a range of pore diameters between about 50 Å to about 10,000 Å and a pore volume between about 0.5 cc/g to about 4.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

In another embodiment, the method of reducing inflammation related complications of (a) cancer or (b) cancer therapy or immunotherapy in a subject in need thereof includes administering a porous biocompatible polymer sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid of the subject. The sorbent may be administered extracorporeally, intravenously, intramuscularly, intratumorally, intradermally, intraperitoneally, subcutaneously, topically, orally, nasally, via a feeding tube, or rectally. The physiologic fluid may be blood.

In another embodiment, the method of reducing inflammation related complications of (a) cancer or (b) cancer therapy or immunotherapy in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer. The physiologic fluid may be blood.

In another embodiment, the method of reducing inflammation related complications of (a) cancer or (b) cancer therapy or immunotherapy in a subject in need thereof includes contacting a physiologic fluid of the subject with a porous biocompatible polymer sorbent comprising a range of pore diameters between about 50 Å to about 10,000 Å and a pore volume between about 0.5 cc/g to about 4.0 cc/g dry polymer. The physiologic fluid may be blood.

In another embodiment, the method of reducing inflammation related complications of (a) cancer or (b) cancer therapy or immunotherapy in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer. The physiologic fluid may be blood.

In an alternate embodiment, the method of reducing inflammation related complications of (a) cancer or (b) cancer therapy or immunotherapy in a subject in need thereof includes providing to the subject a porous biocompatible polymer sorbent comprising a range of pore diameters between about 50 Å to about 3000 Å and a pore volume between about 0.5 cc/g to about 3.0 cc/g dry polymer.

In another embodiment, the method of reducing inflammation related complications of (a) cancer or (b) cancer therapy or immunotherapy in a subject in need thereof includes providing to the subject a porous biocompatible polymer sorbent comprising a range of pore diameters between about 50 Å to about 10,000 Å and a pore volume between about 0.5 cc/g to about 4.0 cc/g dry polymer.

In another alternate embodiment, the method of reducing inflammation related complications of (a) cancer or (b) cancer therapy or immunotherapy in a subject in need thereof includes providing to the subject a porous biocompatible polymer sorbent comprising a range of pore diameters between about 50 Å to about 40,000 Å and a pore volume between about 0.5 cc/g to about 5.0 cc/g dry polymer.

Another embodiment of the invention is a porous biocompatible polymer sorbent comprising a range of pore diameters between about 50 Å to about 3000 Å and a pore volume between about 0.5 cc/g to about 3.0 cc/g dry polymer for use in reducing inflammation related complications of (a) cancer or (b) cancer therapy or immunotherapy. An alternate embodiment is a porous biocompatible polymer sorbent comprising a range of pore diameters between about 50 Å to about 3000 Å and a pore volume between about 0.5 cc/g to about 3.0 cc/g dry polymer for use in the manufacture of a medicament for reducing inflammation related complications of (a) cancer or (b) cancer therapy or immunotherapy.

Another embodiment of the invention is a porous biocompatible polymer sorbent comprising a range of pore diameters between about 50 Å to about 10,000 Å and a pore volume between about 0.5 cc/g to about 4.0 cc/g dry polymer for use in reducing inflammation related complications of (a) cancer or (b) cancer therapy or immunotherapy. An alternate embodiment is a porous biocompatible polymer sorbent comprising a range of pore diameters between about 50 Å to about 10,000 Å and a pore volume between about 0.5 cc/g to about 4.0 cc/g dry polymer for use in the manufacture of a medicament for reducing inflammation related complications of (a) cancer or (b) cancer therapy or immunotherapy.

Another embodiment of the invention is a porous biocompatible polymer sorbent comprising a range of pore diameters between about 50 Å to about 40,000 Å and a pore volume between about 0.5 cc/g to about 5.0 cc/g dry polymer for use in reducing inflammation related complications of (a) cancer or (b) cancer therapy or immunotherapy. An alternate embodiment is a porous biocompatible polymer sorbent comprising a range of pore diameters between about 50 Å to about 40,000 Å and a pore volume between about 0.5 cc/g to about 5.0 cc/g dry polymer for use in the manufacture of a medicament for reducing inflammation related complications of (a) cancer or (b) cancer therapy or immunotherapy.

Other embodiments of the invention are directed to methods of improving the immune function in a subject in need thereof before, during or after treatment with an anticancer agent, anticancer treatment, and/or immunotherapeutic agent using the sorbents of the disclosure. The subject may have cancer. In one embodiment, the methods improve the immune function of the subject before treatment with an anticancer agent, anticancer treatment, and/or immuno-therapeutic agent. In another embodiment, the methods improve the immune function of the subject after treatment with an anticancer agent, anticancer treatment, and/or immunotherapeutic agent. In yet another embodiment, the methods improve the immune function of the subject during treatment with an anticancer agent, anticancer treatment, and/or immunotherapeutic agent. The anticancer agent, anticancer treatment, or immunotherapeutic agent comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

In one embodiment, the method of improving the immune function in a subject in need thereof includes administering a porous biocompatible sorbent to the subject, wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid, such as blood, of the subject. The sorbent may be administered extracorporeally, intravenously, intramuscularly, intratumorally, intradermally, intraperitoneally, subcutaneously, topically, orally, nasally, via a feeding tube, or rectally.

In another embodiment, the method of improving the immune function in a subject in need thereof includes administering a porous biocompatible sorbent to the subject, wherein the polymer sorbent comprises a range of pore diameters between about 50 Å to about 10,000 Å and a pore volume between about 0.5 cc/g to about 4.0 cc/g dry polymer, and wherein the administering places the sorbent in contact with a physiologic fluid, such as blood, of the subject. The sorbent may be administered extracorporeally, intravenously, intramuscularly, intratumorally, intradermally, intraperitoneally, subcutaneously, topically, orally, nasally, via a feeding tube, or rectally.

In another embodiment, the method of improving the immune function in a subject in need thereof includes administering a porous biocompatible sorbent to the subject, wherein the polymer sorbent comprises a range of pore diameters between about 50 Å to about 40,000 Å and a pore volume between about 0.5 cc/g to about 5.0 cc/g dry polymer, and wherein the administering places the sorbent in contact with a physiologic fluid, such as blood, of the subject. The sorbent may be administered extracorporeally, intravenously, intramuscularly, intratumorally, intradermally, intraperitoneally, subcutaneously, topically, orally, nasally, via a feeding tube, or rectally.

In one embodiment, the method of improving the immune function in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer. The contacting may include administering the sorbent intravenously, intramuscularly, intratumorally, intradermally, intraperitoneally, subcutaneously, topically, orally, nasally, via a feeding tube, or rectally. Alternatively, the sorbent may be extracorporeal. In certain embodiments, the physiologic fluid is blood.

In another embodiment, the method of improving the immune function in a subject in need thereof includes contacting a physiologic fluid of the subject with a porous biocompatible polymer sorbent comprising a range of pore diameters between about 50 Å to about 10,000 Å and a pore volume between about 0.5 cc/g to about 4.0 cc/g dry polymer. The contacting may include administering the sorbent intravenously, intramuscularly, intratumorally, intradermally, intraperitoneally, subcutaneously, topically, orally, nasally, via a feeding tube, or rectally. Alternatively, the sorbent may be extracorporeal. In certain embodiments, the physiologic fluid is blood.

In another embodiment, the method of improving the immune function in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer. The contacting may include administering the sorbent intravenously, intramuscularly, intratumorally, intradermally, intraperitoneally, subcutaneously, topically, orally, nasally, via a feeding tube, or rectally. Alternatively, the sorbent may be extracorporeal. In certain embodiments, the physiologic fluid is blood.

In yet another embodiment, the method of improving the immune function in a subject in need thereof includes providing to the subject a porous biocompatible polymer sorbent comprising a range of pore diameters between about 50 Å to about 3000 Å and a pore volume between about 0.5 cc/g to about 3.0 cc/g dry polymer.

In another embodiment, the method of improving the immune function in a subject in need thereof includes providing to the subject a porous biocompatible polymer sorbent comprising a range of pore diameters between about 50 Å to about 10,000 Å and a pore volume between about 0.5 cc/g to about 4.0 cc/g dry polymer.

In another embodiment, the method of improving the immune function in a subject in need thereof includes providing to the subject a porous biocompatible polymer sorbent comprising a range of pore diameters between about 50 Å to about 40,000 Å and a pore volume between about 0.5 cc/g to about 5.0 cc/g dry polymer.

Another embodiment of the invention is a porous biocompatible polymer sorbent comprising a range of pore diameters between about 50 Å to about 3000 Å and a pore volume between about 0.5 cc/g to about 3.0 cc/g dry polymer for use in improving the immune function in a subject in need thereof before, during or after treatment with an anticancer agent, anticancer treatment, and/or immunotherapeutic agent. An alternate embodiment is a porous biocompatible polymer sorbent comprising a range of pore diameters between about 50 Å to about 3000 Å and a pore volume between about 0.5 cc/g to about 3.0 cc/g dry polymer for use in the manufacture of a medicament for improving the immune function in a subject in need thereof before, during or after treatment with an anticancer agent, anticancer treatment, and/or immunotherapeutic agent.

Another embodiment of the invention is a porous biocompatible polymer sorbent comprising a range of pore diameters between about 50 Å to about 10,000 Å and a pore volume between about 0.5 cc/g to about 4.0 cc/g dry polymer for use in improving the immune function in a subject in need thereof before, during or after treatment with an anticancer agent, anticancer treatment, and/or immunotherapeutic agent. An alternate embodiment is a porous biocompatible polymer sorbent comprising a range of pore diameters between about 50 Å to about 10,000 Å and a pore volume between about 0.5 cc/g to about 4.0 cc/g dry polymer for use in the manufacture of a medicament for improving the immune function in a subject in need thereof before, during or after treatment with an anticancer agent, anticancer treatment, and/or immunotherapeutic agent.

Another embodiment of the invention is a porous biocompatible polymer sorbent comprising a range of pore diameters between about 50 Å to about 40,000 Å and a pore volume between about 0.5 cc/g to about 5.0 cc/g dry polymer for use in improving the immune function in a subject in need thereof before, during or after treatment with an anticancer agent, anticancer treatment, and/or immunotherapeutic agent. An alternate embodiment is a porous biocompatible polymer sorbent comprising a range of pore diameters between about 50 Å to about 40,000 Å and a pore volume between about 0.5 cc/g to about 5.0 cc/g dry polymer for use in the manufacture of a medicament for improving the immune function in a subject in need thereof before, during or after treatment with an anticancer agent, anticancer treatment, and/or immunotherapeutic agent.

Alternate embodiments of the invention are directed to methods of improving effectiveness of cancer therapy or immunotherapy in a subject in need thereof using the sorbents of the disclosure. The methods may improve the effectiveness of cancer therapy. Alternately, they may improve the effectiveness of immunotherapy. In certain embodiments, the cancer therapy or immunotherapy includes administration of one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

In one embodiment, the method of improving effectiveness of cancer therapy or immunotherapy in a subject in need thereof includes administering a porous biocompatible sorbent to the subject wherein the polymer sorbent comprises a range of pore diameters between about 50 Å to about 3000 Å and a pore volume between about 0.5 cc/g to about 3.0 cc/g dry polymer, and wherein the administering places the sorbent in contact with a physiologic fluid of the subject. The administration may be extracorporeally, intravenously, intramuscularly, intratumorally, intradermally, intraperitoneally, subcutaneously, topically, orally, nasally, via a feeding tube, or rectally. The biological fluid may be blood.

In another embodiment, the method of improving effectiveness of cancer therapy or immunotherapy in a subject in need thereof includes administering a porous biocompatible sorbent to the subject wherein the polymer sorbent comprises a range of pore diameters between about 50 Å to about 10,000 Å and a pore volume between about 0.5 cc/g to about 4.0 cc/g dry polymer, and wherein the administering places the sorbent in contact with a physiologic fluid of the subject. The administration may be extracorporeally, intravenously, intramuscularly, intratumorally, intradermally, intraperitoneally, subcutaneously, topically, orally, nasally, via a feeding tube, or rectally. The biological fluid may be blood.

In another embodiment, the method of improving effectiveness of cancer therapy or immunotherapy in a subject in need thereof includes administering a porous biocompatible sorbent to the subject wherein the polymer sorbent comprises a range of pore diameters between about 50 Å to about 40,000 Å and a pore volume between about 0.5 cc/g to about 4.0 cc/g dry polymer, and wherein the administering places the sorbent in contact with a physiologic fluid of the subject. The administration may be extracorporeally, intravenously, intramuscularly, intratumorally, intradermally, intraperitoneally, subcutaneously, orally, topically, nasally, via a feeding tube, or rectally. The biological fluid may be blood.

In another embodiment, the method of improving effectiveness of cancer therapy or immunotherapy in a subject in need thereof includes contacting a physiologic fluid of the subject with a porous biocompatible polymer sorbent comprising a range of pore diameters between about 50 Å to about 3000 Å and a pore volume between about 0.5 cc/g to about 3.0 cc/g dry polymer. The sorbent may be extracorporeal. Alternatively, the contacting includes administering the sorbent intravenously, intramuscularly, intratumorally, intradermally, intraperitoneally, subcutaneously, topically, orally, nasally, via a feeding tube, or rectally. The biological fluid may be blood.

In another embodiment, the method of improving effectiveness of cancer therapy or immunotherapy in a subject in need thereof includes contacting a physiologic fluid of the subject with a porous biocompatible polymer sorbent comprising a range of pore diameters between about 50 Å to about 10,000 Å and a pore volume between about 0.5 cc/g to about 4.0 cc/g dry polymer. The sorbent may be extracorporeal. Alternatively, the contacting includes administering the sorbent intravenously, intramuscularly, intratumorally, intradermally, intraperitoneally, subcutaneously, topically, orally, nasally, via a feeding tube, or rectally. The biological fluid may be blood.

In another embodiment, the method of improving effectiveness of cancer therapy or immunotherapy in a subject in need thereof includes contacting a physiologic fluid of the subject with a porous biocompatible polymer sorbent comprising a range of pore diameters between about 50 Å to about 40,000 Å and a pore volume between about 0.5 cc/g to about 5.0 cc/g dry polymer. The sorbent may be extracorporeal. Alternatively, the contacting includes administering the sorbent intravenously, intramuscularly, intratumorally, intradermally, intraperitoneally, subcutaneously, topically, orally, nasally, via a feeding tube, or rectally. The biological fluid may be blood.

In yet another embodiment, the method of improving effectiveness of cancer therapy or immunotherapy in a subject in need thereof includes providing to the subject a porous biocompatible polymer sorbent comprising a range of pore diameters between about 50 Å to about 3000 Å and a pore volume between about 0.5 cc/g to about 3.0 cc/g dry polymer.

In another embodiment, the method of improving effectiveness of cancer therapy or immunotherapy in a subject in need thereof includes providing to the subject a porous biocompatible polymer sorbent comprising a range of pore diameters between about 50 Å to about 10,000 Å and a pore volume between about 0.5 cc/g to about 4.0 cc/g dry polymer.

In another embodiment, the method of improving effectiveness of cancer therapy or immunotherapy in a subject in need thereof includes providing to the subject a porous biocompatible polymer sorbent comprising a range of pore diameters between about 50 Å to about 40,000 Å and a pore volume between about 0.5 cc/g to about 5.0 cc/g dry polymer.

Yet another embodiment of the invention is a porous biocompatible polymer sorbent comprising a range of pore diameters between about 50 Å to about 3000 Å and a pore volume between about 0.5 cc/g to about 3.0 cc/g dry polymer for use in improving effectiveness of cancer therapy or immunotherapy in a subject in need thereof. An alternate embodiment is a porous biocompatible polymer sorbent comprising a range of pore diameters between about 50 Å to about 3000 Å and a pore volume between about 0.5 cc/g to about 3.0 cc/g dry polymer for use in the manufacture of a medicament for improving effectiveness of cancer therapy or immunotherapy.

Another embodiment of the invention is a porous biocompatible polymer sorbent comprising a range of pore diameters between about 50 Å to about 10,000 Å and a pore volume between about 0.5 cc/g to about 4.0 cc/g dry polymer for use in improving effectiveness of cancer therapy or immunotherapy in a subject in need thereof. An alternate embodiment is a porous biocompatible polymer sorbent comprising a range of pore diameters between about 50 Å to about 10,000 Å and a pore volume between about 0.5 cc/g to about 4.0 cc/g dry polymer for use in the manufacture of a medicament for improving effectiveness of cancer therapy or immunotherapy.

Another embodiment of the invention is a porous biocompatible polymer sorbent comprising a range of pore diameters between about 50 Å to about 40,000 Å and a pore volume between about 0.5 cc/g to about 5.0 cc/g dry polymer for use in improving effectiveness of cancer therapy or immunotherapy in a subject in need thereof. An alternate embodiment is a porous biocompatible polymer sorbent comprising a range of pore diameters between about 50 Å to about 40,000 Å and a pore volume between about 0.5 cc/g to about 5.0 cc/g dry polymer for use in the manufacture of a medicament for improving effectiveness of cancer therapy or immunotherapy.

In specific embodiments of the methods or uses, the sorbent sorbs PD-L1.

In specific embodiments of the methods or uses, the sorbent sorbs extracellular vesicles associated with PD-L1.

In any of these methods or uses, the sorbent may comprise a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

As used herein, when the methods refer to administering, the administering encompasses giving the sorbent access to a physiologic fluid. For example, when the fluid is blood access may be achieved by placing sorbent inside a blood vessel or by connecting to a blood vessel.

### Sorbents

The invention also provides for porous polymer sorbents suitable for use in treating cancers and related methods described herein. The porous polymer sorbent is configured to sorb cytokines, interleukins, chemokines, tumor necrosis factor, interferons, lymphokines and other proteins involved in immunoparalysis from a physiologic fluid from a patient. In certain embodiments, the sorbent is configured to remove such proteins from the blood of a patient.

The porous polymer sorbent is also configured to sorb ligands and other proteins involved in immune suppression from a physiologic fluid from a patient. In certain embodiments, the sorbent is configured to remove such proteins from the blood of a patient.

The porous polymer sorbent can also be configured to sorb extracellular vesicles associated with cytokines, interleukins, chemokines, tumor necrosis factor, interferons, lymphokines, ligands and other proteins involved in immunoparalysis or immune suppression from a physiologic fluid from a patient. In certain embodiments, the sorbent is configured to remove such vesicles from the blood of a patient.

The polymer can be incorporated in a pharmaceutical composition comprising the sorbent and a pharmaceutically acceptable excipient or adjuvant. The polymer can be supplied as a slurry, or suspension, or dry powder or other dry particulate capable of being wetted. In some methods, the sorbent is supplied as a slurry or suspension packaged in either single dose or multidose packages for oral administration. In other methods, the sorbent is supplied as a slurry or suspension packaged in either single dose or multidose packages for administration by enema or feeding tube or any combination therein.

The polymer can also be supplied as a dry powder or other dry particulate capable of being wetted externally or internally in the alimentary canal, including in the gastric or enteric environment, with or without the addition of wetting agents such as ethyl or isopropyl alcohol. In yet other embodiments, the polymer is supplied as tablet, dry powder, other dry particulate, capsule, or suppository packaged in bottles or blister packs for administration.

In some methods, the polymer is not metabolizable by mammals, including humans. Such polymers, if administered to a subject, will eventually pass as waste from the subject. For example, sorbent that has bound with a target biomolecule and any unbound sorbent is eventually processed by the kidneys and passes from the body in the subject's urine.

While not wanting to be bound by theory, the treatment is believed to take advantage of the sorbing capacity of the biocompatible polymers (also referred to as sorbents). “Biocompatible” refers to a polymer capable of contact with living tissues or organisms without causing harm during the time that the polymer is in contact with the tissue or organism. In some embodiments, it is intended that the polymer is tolerated by the gut and alimentary canal of the organism. The polymers of the present invention are preferably non-toxic. In some aspects of the presently described methods, administering the sorbent can occur prior to, simultaneously, or subsequent to administration of an immunotherapy or other cancer therapy.

In some embodiments, the polymer has a pore structure such that the total pore volume of pore size in the range of 50 Å to 3000 Å is greater than 0.5 cc/g to 3.0 cc/g dry polymer; wherein the ratio of pore volume between 50 Å to 3,000 Å (pore diameter) to the pore volume between 500 Å to 3,000 Å (pore diameter) of the polymer is smaller than 200:1; and the ratio of pore volume between 50 Å to 3,000 Å in diameter to the pore volume between 1,000 Å to 3,000 Å in diameter of the polymer is greater than 20:1. The said ratios can be alternatively specified in terms of pore surface area (such as the ratio of pore surface area between 50 Å to 3,000 Å to pore surface area between 500 Å to 3,000 Å of the polymer); and therefore is an alternative way of specifying the same pore structure.

In some embodiments, the polymers are coated polymers comprising at least one crosslinking agent and at least one dispersing agent. In some embodiments, the polymers comprise at least one crosslinking agent, and in some embodiments, the polymers comprise at least one dispersing agent. Suitable dispersing agents include hydroxyethyl cellulose, hydroxypropyl cellulose, poly(hydroxyethyl methacrylate), poly(hydroxyethyl acrylate), poly(hydroxypropyl methacrylate), poly(hydroxypropyl acrylate), poly(dimethylaminoethyl methacrylate), poly(dimethylaminoethyl acrylate), poly(diethylamimoethyl methacrylate), poly(diethylaminoethyl acrylate), poly(vinyl alcohol), poly(N-vinylpyrrolidinone), salts of poly(methacrylic acid), and salts of poly(acrylic acid) and mixtures thereof.

In some embodiments, the sorbent comprises at least one crosslinking agent, at least one monomer, at least one dispersing agent and at least one porogen. Porogens that may be used in the invention include one or more of benzyl alcohol, cyclohexane, cyclohexanol, cyclohexanol/toluene mixtures, cyclohexanone, decane, decane/toluene mixtures, di-2-ethylhexylphosphoric acid, di-2-ethylhexyl phthalate, 2-ethyl-1-hexanoic acid, 2-ethyl-1-hexanol, 2-ethyl-1-hexanol/n-heptane mixtures, 2-ethyl-1-hexanol/toluene mixtures, isoamyl alcohol, n-heptane, n-heptane/ethylacetate, n-heptane/isoamyl acetate, n-heptane/tetraline mixtures, n-heptane/toluene mixtures, n-hexane/toluene mixtures, pentanol, poly(styrene-co-methyl methacrylate)/dibutyl phthalate, polystyrene/2-ethyl-1-hexanol mixtures, polystyrene/dibutyl phthalate, polystyrene/n-hexane mixtures, polystyrene/toluene mixtures, toluene, tri-n-butylphosphate, 1,2,3-trichloropropane/2-ethyl-1-hexanol mixtures, 2,2,4-trimethyl pentane (isooctane), trimethyl pentane/toluene mixtures, poly(propylene glycol)/toluene mixtures poly(propylene glycol)/cyclohexanol mixtures, and poly(propylene glycol)/2-ethyl-1-hexanol mixtures.

In certain embodiments, the dispersing agent is one or more of hydroxyethyl cellulose, hydroxypropyl cellulose, poly (hydroxyethyl methacrylate), poly (hydroxyethyl acrylate), poly (hydroxypropyl methacrylate), poly (hydroxypropyl acrylate), poly (dimethylaminoethyl methacrylate), poly (dimethylaminoethyl acrylate), poly (diethylamimoethyl methacrylate), poly (diethylaminoethyl acrylate), poly (vinyl alcohol), poly (N-vinylpyrrolidinone), salts of poly (methacrylic acid), or salts of poly(acrylic acid).

Suitable crosslinking agents include divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, divinylformamide and mixtures thereof. In some aspects, the polymer is developed simultaneously with the formation of the coating, such that the dispersing agent gets chemically bound to the surface of the polymer.

In some aspects, the polymer is derived from one or more monomers selected from divnylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

In some aspects, the polymer is an ion exchange polymer. These polymers include crosslinked polystyrene including those containing some divinylbenzene residues. The polymers are typically porous beads, and such polymers may comprise acidic or basic functional groups. Suitable functional groups include sulfonic acid groups, amine groups, carboxylic acid groups, and salts of any of the forgoing groups.

In some other aspects of the present disclosure, the polymer is a cellulosic polymer. Suitable polymers include cross-linked dextran gels such as Sephadex®.

The polymer according to some aspects of the present disclosure is a porous, highly crosslinked styrene or divinylbenzene copolymer. In some aspects, the polymer is a macroporous or mesoporous styrene-divinylbenzene-ethylstyrene copolymer subjected to a partial chloromethylation to a chlorine content of up to 7% molecular weight. In other aspects the polymer is a hypercrosslinked polystyrene produced from crosslinked styrene copolymers by an extensive chloromethylation and a subsequent post-crosslinking by treating with a Friedel-Crafts catalyst in a swollen state. In yet other aspects, the polymer is a hypercrosslinked polystyrene produced from crosslinked styrene copolymers by an extensive additional post-crosslinking in a swollen state with bifunctional crosslinking agents selected from the group comprising of monochlorodimethyl ether and p-xylilene dichloride.

In some aspects, a polymer useful in the practice of the invention is a hydrophilic self-wetting polymer that can be administered as a dry powder containing hydrophilic functional groups such as, amines, hydroxyl, sulfonate, and carboxyl groups.

In some aspects, a polymer useful in the invention is a macroporous polymer prepared from the polymerizable monomers of styrene, divinylbenzene, ethylvinylbenzene, and the acrylate and methacrylate monomers such as those listed below by manufacturer Rohm and Haas Company, (now part of Dow Chemical Company): (i) macroporous polymeric sorbents such as Amberlite™ XAD-1, Amberlite™ XAD-2, Amberlite™ XAD-4, Amberlite™ XAD-7, Amberlite™ XAD-7HIP, Amberlite™ XAD-8, Amberlite™ XAD-16, Amberlite™ XAD-16 HP, Amberlite™ XAD-18, Amberlite™ XAD-200, Amberlite™ XAD-1180, Amberlite™ XAD-2000, Amberlite™ XAD-2005, Amberlite™ XAD-2010, Amberlite™ XAD-761, and Amberlite™ XE-305, and chromatographic grade sorbents such as Amberchrom™ CG 71,s,m,c, Amberchrom™ CG 161,s,m,c, Amberchrom™ CG 300,s,m,c, and Amberchrom™ CG 1000,s,m,c. Dow Chemical Company: Dowex® Optipore™ L-493, Dowex® Optipore™ V-493, Dowex® Optipore™ V-502, Dowex® Optipore™ L-285, Dowex® Optipore™ L-323, and Dowex® Optipore™ V-503. Lanxess (formerly Bayer and Sybron): Lewatit® VPOC 1064 MD PH, Lewatit® VPOC 1163, Lewatit® OC EP 63, Lewatit® S 6328 Å, Lewatit® OC 1066, and Lewatit® 60/150 MIBK. Mitsubishi Chemical Corporation: Diaion® HP 10, Diaion® HP 20, Diaion® HP 21, Diaion® HP 30, Diaion® HP 40, Diaion® HP 50, Diaion® SP70, Diaion® SP 205, Diaion® SP 206, Diaion® SP 207, Diaion® SP 700, Diaion® SP 800, Diaion® SP 825, Diaion® SP 850, Diaion® SP 875, Diaion® HP 1MG, Diaion® HP 2MG, Diaion® CHP 55 Å, Diaion® CHP 55Y, Diaion® CHP 20 Å, Diaion® CHP 20Y, Diaion® CHP 2MGY, Diaion® CHP 20P, Diaion® HP 20SS, Diaion® SP 20SS, and Diaion® SP 207SS. Purolite Company: Purosorb™ AP 250 and Purosorb™ AP 400.

### Aspects of the Invention

The invention is illustrated by the following non-limiting aspects.

Aspect 1. A method of treating cancer in a subject, comprising treating a therapeutically effective amount of a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 2. A method of treating cancer in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer;
and
wherein the administering places the sorbent in contact with a physiologic fluid of the subject.

Aspect 3. The method of aspect 1 or aspect 2, wherein the sorbent is administered extracorporeally.

Aspect 4. The method of aspect 1 or aspect 2, wherein the sorbent is administered intravenously, intramuscularly, intratumorally, intradermally, intraperitoneally, subcutaneously, topically, orally, nasally, via a feeding tube, or rectally.

Aspect 5. A method of treating cancer in a subject in need thereof, comprising contacting a physiologic fluid of the subject with a porous biocompatible polymer sorbent, wherein
the sorbent comprises a range of pore diameters between about 50 Å to about 3000 Å and
a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 6. The method of aspect 5, wherein the sorbent is administered intravenously, intramuscularly, intratumorally, intradermally, intraperitoneally, subcutaneously, topically, orally, nasally, via a feeding tube, or rectally.

Aspect 7. The method of aspect 5, wherein the sorbent is extracorporeal.

Aspect 8. The method of any one of aspects 2-7, wherein the physiologic fluid is blood.

Aspect 9. A method of treating cancer in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 10. The method of any one of aspects 1-9, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

Aspect 11. The method of any one of aspects 1-10, wherein the sorbent is produced using at least one crosslinking agent and at least one monomer.

Aspect 12. The method of aspect 11, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

Aspect 13. The method of aspect 11 or aspect 12, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

Aspect 14. The method of any one of aspects 11-13, wherein the sorbent is produced additionally utilizing at least one dispersing agent and at least one porogen.

Aspect 15. The method of any one of aspects 1-14, wherein the sorbent comprises a biocompatible and hemocompatible exterior coating that is covalently bound to the sorbent by free-radical grafting.

Aspect 16. The method of any one of aspects 1-15, wherein said sorbent is administered in combination with an anticancer agent, anticancer treatment, or an immunotherapeutic agent.

Aspect 17. The method of aspect 16, wherein said sorbent is administered prior to or at the same time as said anticancer agent, anticancer treatment, or immunotherapeutic agent

Aspect 18. The method of aspect 16, wherein said sorbent is administered following administration of the anticancer agent, anticancer treatment, or immunotherapeutic agent.

Aspect 19. The method of aspect 17 or aspect 18, wherein the anticancer agent, anticancer treatment, or immunotherapeutic agent comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

Aspect 20. The method of any one of aspects 1-19, wherein the sorbent reduces the concentration of one or more of cytokines, inflammatory mediators, toxins, electrolytes, chemicals, cellular debris, and metabolic waste products in the patient relative to an untreated subject.

Aspect 21. The method of any one of aspects 1-20, wherein the method reduces the entry of CAR-T cells and other activated cells into the central nervous system relative to an untreated subject.

Aspect 22. The method of any one of aspects 1-20, wherein the method reduces inhibition of the immune system.

Aspect 23. The method of any one of aspects 1-20, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

Aspect 24. The method of any one of aspects 1-23, wherein administration of the sorbent reduces inhibitors of the immune system before, during or after administration of the anticancer agent, anticancer treatment, or immunotherapeutic agent.

Aspect 25. The method of any one of aspects 1-23, wherein administration of the sorbent results in removal of one or more of a) cytokine or chemokine, b) soluble checkpoint molecule, and c) extracellular vesicle associated with a checkpoint molecule from bodily fluid of the subject.

Aspect 26. The method of aspect 25, wherein the soluble or extracellular vesicle-associated checkpoint molecule is one or more of the examples found in Table I.

Aspect 27. The method of aspect 25, wherein the cytokine or chemokine comprises one or more of a member of the interleukin, interferon, tumor growth factor, or tumor necrosis factor family.

Aspect 28. The method of any one of aspects 1-27, wherein the sorbent comprises one or more residues of divinylbenzene and ethylvinylbenzene, styrene, and ethylstyrene monomers.

Aspect 29. A method of reducing inflammation related complications of (a) cancer or (b) cancer therapy or immunotherapy in a subject comprising treating the subject with a therapeutically effective amount of a porous biocompatible polymer sorbent, wherein the sorbent comprises a range of pore diameters between about 50 Å to about 3000 Å and a pore volume between about 0.5 cc/g to about 3.0 cc/g dry polymer; or the sorbent comprises a range of pore diameters between about 50 Å to about 10,000 Å and a pore volume between about 0.5 cc/g to about 4.0 cc/g dry polymer; or the sorbent comprises a range of pore diameters between about 50 Å to about 40,000 Å and a pore volume between about 0.5 cc/g to about 5.0 cc/g dry polymer.

Aspect 30. A method of reducing inflammation related complications of (a) cancer or (b) cancer therapy or immunotherapy in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer;
and
wherein the administering places the sorbent in contact with a physiologic fluid of the subject.

Aspect 31. The method of aspect 29 or aspect 30, wherein the sorbent is administered extracorporeally.

Aspect 32. The method of aspect 29 or aspect 30, wherein the sorbent is administered intravenously, intramuscularly, intratumorally, intradermally, intraperitoneally, subcutaneously, topically, orally, nasally, via a feeding tube, or rectally.

Aspect 33. A method of reducing inflammation related complications of (a) cancer or (b) cancer therapy or immunotherapy in a subject in need thereof comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 34. The method of aspect 33, wherein the contacting comprises administering the sorbent intravenously, intramuscularly, intratumorally, intradermally, intraperitoneally, subcutaneously, topically, orally, nasally, via a feeding tube, or rectally.

Aspect 35. The method of aspect 33, wherein the sorbent is extracorporeal.

Aspect 36. The method of any one of aspects 30-35, wherein the physiologic fluid is blood.

Aspect 37. A method of reducing inflammation related complications of (a) cancer or (b) cancer therapy or immunotherapy in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 38. The method of any one of aspects 29-37, wherein the method reduces inflammation related complication of cancer.

Aspect 39. The method of any one of aspects 29-37, wherein the method reduces inflammation due to cancer therapy.

Aspect 40. The method of any one of aspects 29-37, wherein the method reduces inflammation due to immunotherapy.

Aspect 41. The method of aspect 39 or aspect 40, wherein the cancer therapy or immunotherapy comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

Aspect 42. The method of any one of aspects 38-40, wherein reducing inflammation improves one or more of the manifestation, severity, or progression of cachexia.

Aspect 43. The method of any one of aspects 29-42, wherein the method reduces the entry of CAR-T cells and other activated cells into the central nervous system relative to an untreated subject.

Aspect 44. The method of any one of aspects 29-42, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

Aspect 45. The method of any one of aspects 29-42, wherein the sorbent reduces the concentration of one or more of cytokines, chemokines, inflammatory mediators, toxins, electrolytes, chemicals, cellular debris, and metabolic waste products in the patient relative to an untreated subject.

Aspect 46. The method of any one of aspects 29-45, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 to 10,000 Å (pore diameter) of the sorbent is smaller than 2:1.

Aspect 47. The method of any one of aspects 29-46, wherein the sorbent is produced using at least one crosslinking agent and at least one monomer.

Aspect 48. The method of aspect 47, wherein the monomer comprises divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

Aspect 49. The method of aspect 47 or aspect 48, wherein the crosslinking agent comprises one or more or divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

Aspect 50. The method of any one of aspects 47-49, wherein the sorbent is produced additionally utilizing at least one dispersing agent and at least one porogen.

Aspect 51. The method of any one of aspects 29-50, wherein the sorbent comprises a biocompatible and hemocompatible exterior coating that is covalently bound to the sorbent by free-radical grafting.

Aspect 52. The method of any one of aspects 29-51, wherein the sorbent comprises one or more residues of divinylbenzene and ethylvinylbenzene, styrene, and ethylstyrene monomers.

Aspect 53. A method of improving immune system function in a subject before, during or after treatment with an anticancer agent, anticancer treatment, and/or immunotherapeutic agent, comprising administering to the subject a therapeutically effective amount of a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 54. A method of improving immune system function in a subject in need thereof before, during or after treatment with an anticancer agent, anticancer treatment, and/or immunotherapeutic agent comprising administering a porous biocompatible sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer;
and
wherein the administering places the sorbent in contact with a physiologic fluid of the subject.

Aspect 55. The method of aspect 53 or aspect 54, wherein the sorbent is administered extracorporeally.

Aspect 56. The method of aspect 53 or aspect 54, wherein the sorbent is administered intravenously, intramuscularly, intratumorally, intradermally, intraperitoneally, subcutaneously, topically, orally, nasally, via a feeding tube, or rectally.

Aspect 57. A method of improving immune system function in a subject in need thereof before, during or after treatment with an anticancer agent, anticancer treatment, or immunotherapeutic agent comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 58. The method of aspect 57, wherein the contacting comprises administering the sorbent intravenously, intramuscularly, intratumorally, intradermally, intraperitoneally, subcutaneously, topically, orally, nasally, via a feeding tube, or rectally.

Aspect 59. The method of aspect 57, wherein the sorbent is extracorporeal.

Aspect 60. The method of any one of aspects 54-59, wherein the physiologic fluid is blood.

Aspect 61. A method of improving immune system function in a subject in need thereof before, during or after treatment with an anticancer agent, anticancer treatment, or immunotherapeutic agent comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 62. The method of any one of aspects 53-61, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

Aspect 63. The method of any one of aspects 53-62, wherein the sorbent is produced using at least one crosslinking agent and at least one monomer.

Aspect 64. The method of aspect 63, wherein the monomer comprising divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

Aspect 65. The method of aspect 63 or aspect 64, wherein the crosslinking agent comprising one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, or divinylformamide.

Aspect 66. The method of any one of aspects 63-65, wherein the sorbent is produced additionally utilizing at least one dispersing agent and at least one porogen.

Aspect 67. The method of any one of aspects 53-66, wherein the sorbent comprises a biocompatible and hemocompatible exterior coating that is covalently bound to the sorbent by free-radical grafting.

Aspect 68. The method of any one of aspects 53-67, wherein the sorbent comprises one or more residues of divinylbenzene and ethylvinylbenzene, styrene, and ethylstyrene monomers.

Aspect 69. The method of any one of aspects 53-68, wherein the anticancer agent, anticancer treatment, or immunotherapeutic agent comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

Aspect 70. The method of any one of aspects 53-69, wherein the method improves immune system function in a subject in need thereof before treatment with an anticancer agent, anticancer treatment, or immunotherapeutic agent.

Aspect 71. The method of any one of aspects 53-69, wherein the method improves immune system function in a subject in need thereof during treatment with an anticancer agent, anticancer treatment, or immunotherapeutic agent.

Aspect 72. The method of any one of aspects 53-69, wherein the method improves immune system function in a subject in need thereof after treatment with an anticancer agent, anticancer treatment, or immunotherapeutic agent.

Aspect 73. The method of one or more of aspects 70-72, wherein administration of the sorbent results in removal of one or more of a) cytokine or chemokine, b) soluble checkpoint molecule, and c) extracellular vesicle associated with a checkpoint molecule from bodily fluid of the subject.

Aspect 74. The method of aspect 73, wherein the soluble or extracellular vesicle-associated checkpoint molecule is one or more of the examples found in Table I.

Aspect 75. The method of aspect 73, wherein the cytokine or chemokine comprises one or more of a member of the interleukin, interferon, tumor growth factor, or tumor necrosis factor family.

Aspect 76. The method of any one of aspects 53, 54, 57, or 61, wherein the improved immune system function includes a reduction in systemic or local immune suppression.

Aspect 77. The method of any one of aspects 53, 54, 57, or 61, wherein the improved immune system function includes a reduction in systemic or local hyperinflammation.

Aspect 78. A method of improving effectiveness of cancer therapy or immunotherapy in a subject, comprising treating the subject with a therapeutically effective amount of a porous biocompatible polymer sorbent, wherein the sorbent comprises a range of pore diameters between about 50 Å to about 3000 Å and a pore volume between about 0.5 cc/g to about 3.0 cc/g dry polymer; or the sorbent comprises a range of pore diameters between about 50 Å to about 10,000 Å and a pore volume between about 0.5 cc/g to about 4.0 cc/g dry polymer; or the sorbent comprises a range of pore diameters between about 50 Å to about 40,000 Å and a pore volume between about 0.5 cc/g to about 5.0 cc/g dry polymer.

Aspect 79. A method of improving effectiveness of cancer therapy or immunotherapy in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer;
and
wherein the administering places the sorbent in contact with a physiologic fluid of the subject.

Aspect 80. The method of aspect 78 or aspect 79, wherein the sorbent is administered extracorporeally.

Aspect 81. The method of aspect 78 or aspect 79, wherein the sorbent is administered intravenously, intramuscularly, intratumorally, intradermally, intraperitoneally, subcutaneously, topically, orally, nasally, via a feeding tube, or rectally.

Aspect 82. A method of improving effectiveness of cancer therapy or immunotherapy in a subject in need thereof comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 83. The method of aspect 82, wherein the contacting comprises administering the sorbent intravenously, intramuscularly, intratumorally, intradermally, intraperitoneally, subcutaneously, topically, orally, nasally, via a feeding tube, or rectally.

Aspect 84. The method of aspect 82 wherein the sorbent is extracorporeal.

Aspect 85. The method of any one of aspects 79-84, wherein the physiologic fluid is blood.

Aspect 86. A method of improving effectiveness of cancer therapy or immunotherapy in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

Aspect 87. The method of any one of aspects 78-86, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

Aspect 88. The method of any one of aspects 78-87, wherein the sorbent is produced using at least one crosslinking agent and at least one monomer.

Aspect 89. The method of aspect 88, wherein the monomer comprising divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

Aspect 90. The method of aspect 88 or aspect 89, wherein the crosslinking agent comprising one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, or divinylformamide.

Aspect 91. The method of any one of aspects 88-90, wherein the sorbent is produced additionally utilizing at least one dispersing agent and at least one porogen.

Aspect 92. The method of any one of aspects 78-91, wherein the sorbent comprises a biocompatible and hemocompatible exterior coating that is covalently bound to the sorbent by free-radical grafting.

Aspect 93. The method of any one of aspects 78-92 wherein the sorbent comprises one or more residues of divinylbenzene and ethylvinylbenzene, styrene, and ethylstyrene monomers.

Aspect 94. The method of any one of aspects 78-93, wherein the cancer therapy or immunotherapy comprises administration of one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

Aspect 95. The method of any one of aspects 78-94, wherein the method improves the effectiveness of cancer therapy.

Aspect 96. The method of any one of aspects 78-94, wherein the method improves the effectiveness of immunotherapy.

Aspect 97. The method of any one of aspects 78-96, wherein the sorbent reduces the concentration of one or more of cytokines, chemokines, inflammatory mediators, toxins, electrolytes, chemicals, cellular debris, and metabolic waste products in the patient relative to an untreated subject.

Aspect 98. The method of any one of aspects 78-96, wherein the method reduces the entry of CAR-T cells and other activated cells into the central nervous system relative to an untreated subject.

Aspect 99. The method of any one of aspects 78-96, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

Aspect 100. The method of any one of aspects 78-96, wherein the method improves one or more of the manifestation, severity, or progression of cachexia.

Aspect 101. The method of one or more of aspects 78-96, wherein administration of the sorbent results in removal of one or more of a) cytokine or chemokine, b) soluble checkpoint molecule, and c) extracellular vesicle associated with a checkpoint molecule from bodily fluid of the subject.

Aspect 102. The method of aspect 101, wherein the soluble or extracellular vesicle-associated checkpoint molecule is one or more of the examples found in Table I.

Aspect 103. The method of aspect 101, wherein the cytokine or chemokine comprises one or more of a member of the interleukin, interferon, tumor growth factor, or tumor necrosis factor family.

### EXAMPLES

The following examples are intended to be exemplary and non-limiting. The following examples of the invention are to further illustrate the nature of the invention. It is believed that one of ordinary skill in the art can, using the preceding description and the following illustrative examples, make and utilize the present invention and practice the claimed methods. It should be understood that the following examples do not limit the invention and that the scope of the invention is to be determined by the appended claims.

### Example 1: Base Sorbent Synthesis CY14175 & CY15077

Reactor Setup: a 4-neck glass lid was affixed to a 3L jacketed cylindrical glass reaction vessel using a stainless-steel flange clamp and PTFE gasket. The lid was fitted with a PTFE stirrer bearing, RTD adapter, and water-cooled reflux condenser. A stainless-steel stirring shaft having five 60° agitators was fit through the stirrer bearing and inserted into a digital overhead stirrer. An RTD was fit through the corresponding adapter and connected to a PolyStat circulating heating and chilling unit. Compatible tubing was used to connect the inlet and outlet of the reaction vessel jacket to the appropriate ports on the PolyStat. The unused port in the lid was used for charging the reactor and was plugged at all other times.

Polymerization: Aqueous phase and organic phase compositions are shown below, in Table II and Table III, respectively. Ultrapure water was split into approximately equal parts in two separate Erlenmeyer flasks, each containing a PTFE coated magnetic stir bar. Poly (vinyl alcohol) (PVA), having a degree of hydrolysis of 85.0 to 89.0 mol percent and a viscosity of 23.0 to 27.0 cP in a 4% aqueous solution at 20° C., was dispersed into the water in the first flask and heated to 80° C. on a hot plate with agitation. Salts (see Table II, MSP, DSP, TSP and Sodium nitrite) were dispersed into the water in the second flask and heated to 80° C. on a hot plate with agitation. Circulation of heat transfer fluid from the PolyStat through the reaction vessel jacket was started, and fluid temperature heated to 60° C. Once PVA and salts dissolved, both solutions were charged to the reactor, one at a time, using a glass funnel. The digital overhead stirrer was powered on, and the rpm set to a value to form appropriate droplet sizes upon organic phase addition. Temperature of the aqueous phase in the kettle was set to 70° C. The organic phase was prepared by adding benzoyl peroxide (BPO) to the divinylbenzene (DVB) in a 2L Erlenmeyer flask and swirling until completely dissolved. 2,2,4-trimethylpentane and toluene were added to the flask, which was swirled to mix well. Once the temperature of the aqueous phase in the reactor reached 70° C., the organic phase was charged into the reactor using a narrow-necked glass funnel. Temperature of the reaction volume dropped upon the organic addition. A temperature program for the PolyStat was started, heating the reaction volume from 60 to 77° C. over 30 minutes, 77 to 80° C. over 30 minutes, holding the temperature at 80° C. for 960 minutes, and cooling to 20° C. over 60 minutes.

TABLE II
Aqueous Phase Composition

| Reagent | Mass (g) |
|---|---|
| Ultrapure water | 1500.000 |
| Poly (vinyl alcohol) (PVA) | 4.448 |
| Monosodium phosphate (MSP) | 4.602 |
| Disodium phosphate (DSP) | 15.339 |
| Trisodium phosphate (TSP) | 9.510 |
| Sodium nitrite | 0.046 |
| Total | 1533.899 |

TABLE III
Organic Phase Compositions

| | CY14175 | CY15077 |
|---|---|---|
| Reagent | Mass (g) | Mass (g) |
| Divinylbenzene, 63% (DVB) | 508.751 | 498.383 |
| 2,2,4-trimethylpentane (Isooctane) | 384.815 | 482.745 |
| Toluene | 335.004 | 222.404 |
| Benzoyl peroxide, 98% (BPO) | 3.816 | 3.738 |
| Total (excluding BPO) | 1228.571 | 1203.532 |

Work-up: reaction volume level in the reactor was marked. Overhead stirrer agitation was stopped, residual liquid siphoned out of the reactor, and the reactor filled to the mark with ultrapure water at room temperature. Overhead stirrer agitation was restarted and the slurry heated to 70° C. as quickly as possible. After 30 minutes, agitation was stopped, and residual liquid siphoned out. Polymer beads were washed five times in this manner. During the final wash, the slurry temperature was cooled to room temperature. After the final water wash, polymer beads were washed with 99% isopropyl alcohol (IPA) in the same manner. 99% IPA was siphoned out and replaced with 70% IPA before transferring the slurry into a clean 4L glass container. Unless noted otherwise, on an as-needed basis the polymer was steam stripped in a stainless-steel tube for 8 hours, rewet in 70% IPA, transferred into DI water, sieved to obtain only the portion of beads having diameters between 300 and 600 m, and dried at 100° C. until no further weight loss on drying was observed.

Cumulative pore volume data for polymers CY14175 and CY15077, measured by nitrogen desorption isotherm and mercury intrusion porosimetry, respectively, are shown below in Tables IV and V, respectively.

TABLE IV
Nitrogen Desorption Isotherm Data for CY14175

| Pore Diameter | Pore size | Cumulative Pore |
|---|---|---|
| Range (Å) | Diameter (Å) | Volume (cm³/g) |
| 1411.9-1126.5 | 1236.809577 | 0.018062878 |
| 1126.5-981.7 | 1043.979923 | 0.038442381 |
| 981.7-752.9 | 836.7828769 | 0.141559621 |
| 752.9-659.9 | 700.1024343 | 0.24336622 |
| 659.9-572.0 | 609.4657394 | 0.416511969 |
| 572.0-483.1 | 519.8089977 | 0.646318614 |
| 483.1-449.8 | 465.2234212 | 0.730406771 |
| 449.8-401.4 | 422.7246485 | 0.849167577 |
| 401.4-354.1 | 374.6289335 | 0.956165766 |
| 354.1-337.9 | 345.6019761 | 0.997336398 |
| 337.9-313.5 | 324.758962 | 1.0547802 |
| 313.5-290.8 | 301.2432086 | 1.09667858 |
| 290.8-262.8 | 275.299967 | 1.164042391 |
| 262.8-247.2 | 254.510376 | 1.199751164 |
| 247.2-233.6 | 240.0176376 | 1.228796957 |
| 233.6-220.1 | 226.435352 | 1.256631669 |
| 220.1-208.6 | 213.9982044 | 1.283063762 |
| 208.6-130.5 | 151.2300725 | 1.464027373 |
| 130.5-105.7 | 115.2567614 | 1.527062065 |
| 105.7-82.8 | 91.14860242 | 1.592486039 |
| 82.8-67.6 | 73.42901881 | 1.641003444 |
| 67.6-57.5 | 61.59836256 | 1.6763711 |
| 57.5-51.6 | 54.15491457 | 1.699539142 |
| 51.6-45.0 | 47.72291376 | 1.728282889 |
| 45.0-39.8 | 42.01726183 | 1.752728216 |
| 39.8-35.8 | 37.55877213 | 1.779016164 |
| 35.8-31.8 | 33.51596841 | 1.8086605 |
| 31.8-28.7 | 30.02327371 | 1.82963357 |
| 28.7-26.0 | 27.18773181 | 1.850084632 |
| 26.0-23.3 | 24.46989555 | 1.87529426 |
| 23.3-20.9 | 21.92055755 | 1.902736527 |
| 20.9-18.5 | 19.52461159 | 1.935789448 |
| 18.5-16.2 | 17.16324429 | 1.97779901 |

TABLE V
Mercury Intrusion Data for CY15077

| Pore size | Cumulative |
|---|---|
| Diameter (Å) | Intrusion (mL/g) |
| 226299.0625 | 3.40136E−30 |
| 213166.0781 | 0.001678752 |
| 201295.1563 | 0.002518128 |
| 172635.8125 | 0.004364755 |
| 139538.0625 | 0.007554384 |
| 113120.7813 | 0.011919139 |
| 90542.36719 | 0.01645177 |
| 78733.25781 | 0.0203129 |
| 72446.375 | 0.022327403 |
| 60340.40234 | 0.027867284 |
| 48343.83984 | 0.035327822 |
| 39009.13672 | 0.040918175 |
| 32136.4082 | 0.04899035 |
| 25330.65625 | 0.063195683 |
| 20981.51563 | 0.079529688 |
| 16219.86426 | 0.108860672 |
| 13252.41211 | 0.141730919 |
| 10501.53613 | 0.193969816 |
| 8359.911133 | 0.262399256 |
| 6786.30127 | 0.345866203 |
| 5538.122559 | 0.438174427 |
| 4337.931152 | 0.563276172 |
| 3501.674805 | 0.681870878 |
| 2838.742188 | 0.804727197 |
| 2593.016846 | 0.865813017 |
| 2266.688965 | 0.938610673 |
| 1831.041748 | 1.056586146 |
| 1509.850708 | 1.163395643 |
| 1394.006104 | 1.21002543 |
| 1294.780151 | 1.257248282 |
| 1207.692627 | 1.293158531 |
| 1131.860962 | 1.326992273 |
| 1065.099976 | 1.35812819 |
| 953.1816406 | 1.405935764 |
| 884.0358887 | 1.445426106 |
| 823.5491333 | 1.478719592 |
| 770.9108276 | 1.510579824 |
| 722.4724731 | 1.537048101 |
| 684.6119995 | 1.564400196 |
| 672.187561 | 1.581117511 |
| 636.7885742 | 1.60271585 |
| 604.7248535 | 1.621845484 |
| 558.1287231 | 1.651492 |
| 518.2624512 | 1.678913713 |
| 483.5536499 | 1.708594561 |
| 453.5110779 | 1.735918999 |
| 426.9998474 | 1.755934 |
| 403.1251526 | 1.783603072 |
| 382.7776794 | 1.793849826 |
| 362.7162476 | 1.817784309 |
| 342.3734436 | 1.838774562 |
| 330.1105042 | 1.851493955 |
| 315.5238037 | 1.869742155 |
| 302.2973938 | 1.885128617 |
| 290.2946777 | 1.895119786 |
| 279.1246643 | 1.912378907 |
| 268.7442627 | 1.924305081 |
| 259.1106873 | 1.936048627 |
| 241.8737793 | 1.955100656 |
| 226.7678223 | 1.972970247 |
| 213.3626251 | 1.988123298 |
| 201.4908142 | 2.007521152 |
| 194.9888611 | 2.022114754 |
| 188.9506989 | 2.033871174 |
| 180.582901 | 2.035052776 |
| 172.8530121 | 2.050720692 |
| 164.9621735 | 2.062945843 |
| 157.8110657 | 2.071056128 |
| 151.1540375 | 2.082133055 |
| 143.9185333 | 2.096480608 |
| 138.4670563 | 2.106938839 |
| 132.8492737 | 2.119287968 |
| 129.5760345 | 2.126605988 |
| 126.5438614 | 2.126605988 |
| 124.2635574 | 2.132267475 |
| 120.8976135 | 2.141504765 |
| 117.3792267 | 2.150759459 |
| 114.791893 | 2.154810667 |
| 111.9475937 | 2.162935257 |
| 108.8830032 | 2.167646885 |
| 106.6480179 | 2.174062729 |
| 104.5217743 | 2.179908991 |
| 102.4295197 | 2.179908991 |
| 100.1580353 | 2.182951927 |
| 98.29322052 | 2.184018135 |
| 96.44822693 | 2.191127539 |
| 94.42159271 | 2.198545218 |
| 91.52587891 | 2.209161043 |
| 89.25807953 | 2.209312439 |
| 87.0777359 | 2.215425491 |
| 85.42358398 | 2.221472025 |
| 83.62612915 | 2.232139587 |
| 82.11174011 | 2.237514496 |
| 79.91614532 | 2.239231586 |
| 78.01462555 | 2.239560127 |
| 76.19993591 | 2.239560127 |
| 75.09249115 | 2.239560127 |
| 73.41201019 | 2.239560127 |
| 72.23709869 | 2.240245819 |
| 71.09960175 | 2.242422104 |
| 69.86301422 | 2.243849993 |
| 68.40761566 | 2.257676363 |
| 67.13697815 | 2.259181261 |
| 66.03359222 | 2.266284466 |
| 65.08189392 | 2.270181179 |
| 64.04368591 | 2.272682428 |
| 62.38490295 | 2.280714512 |
| 61.32764053 | 2.280714512 |
| 60.30379868 | 2.287917852 |
| 59.41370392 | 2.287917852 |
| 58.54679489 | 2.293802738 |
| 57.79866409 | 2.297607183 |
| 56.88977814 | 2.299046278 |
| 55.9213295 | 2.302111387 |
| 54.98665237 | 2.303381443 |

### Example 2: Removal of Soluble PD-L1 (PD-L1) from Bovine Whole Blood

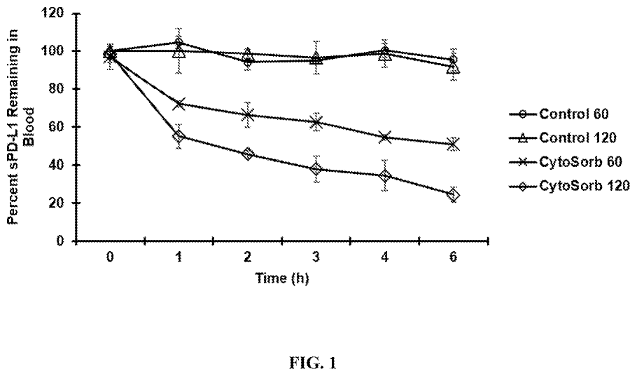

Data from bench top evaluations demonstrates that CytoSorb removes soluble PD-L1 (sPD-L1) from bovine whole blood, as shown in FIG. 1. In this study, two volumes of CytoSorb were evaluated: 60 mL and 120 mL. A starting PD-L1 concentration of 1.4 ng/mL in 800 mL of whole bovine blood was used. Blood was recirculated through the CytoSorb device at a rate of 60 mL/min. After 6 hours, CytoSorb 60 mL removed 49% of the starting sPD-L1 concentration, and CytoSorb 120 mL removed 76% of the starting sPD-L1 concentration. Controls (sham, no CytoSorb) were evaluated at the same time as each CytoSorb experiment. Each group was repeated 3 times (n=3).

As noted, soluble PD-L1 and soluble PD-L2 ligands activate the PD-1 receptor on cytotoxic T-cells, which downregulates T-cell activity and prevents cell-mediated killing of the cancers. Accordingly, this example demonstrates that the biocompatible polymer sorbents of the invention may be used to treat cancer.

### Example 3: Demonstrating Antitumor Efficacy of a Porous Biocompatible Polymer Sorbent by Intratumor or Subcutaneous Injection in a Small Animal Solid Tumor Model

An appropriate small animal model is injected with a cancer cell type under test to induce tumor growth. After the tumors have grown, the animals are then intratumorally injected with the porous polymer beads suspended in appropriate matrix (e.g., saline) and blood samples are taken at various time points to be analyzed for reduction in cytokines and in soluble or extracellular vesicle-associated checkpoint molecules. The tumors are also observed to detect a reduction in tumor growth. Animal body weight changes are compared to the non-treated animals.

For example, C57BL/6 mice are injected with B16-F10 murine tumor cells (0.5×10⁶) in saline. After tumor growth is established, tumor regions are injected subcutaneously with a porous biocompatible polymer sorbent (1 mL, suspended in saline), or saline alone (control). Animal body weight is recorded on day 1 prior to dosing and on days 3, 5, 7 and 9. On days 1, 3, 5, 7 and 9 post cell inoculation, 6 animals in each treatment group are sacrificed for cardiac blood puncture to collect a maximum volume of blood (0.6 to 1.0 mL) and plasma is separated and analyzed for cytokines (e.g. MCP-1, GM-CSF, IFN-b, IFN-g, IL-1a, IL-1b, IL-6, IL-10, IL-12 (p70), IL-17A, IL-23, IL-27, TNF-a) and soluble checkpoint molecules (e.g. sPD-L1) by using appropriate ELISA/multiplex assays. Extracellular vesicle (EV) associated checkpoint molecules (e.g., EV associated PD-L1) are also analyzed after collection of EVs using standard procedures. Additionally, on certain days (e.g., 1, 3, 5, 7 and 9), lungs are harvested and fixed in Bouin's solution for histopathology, pictures of lungs are taken, and metastatic lesions counted.

### Example 4: Demonstrating Antitumor Efficacy of a Porous Biocompatible Polymer Sorbent by Extracorporeal Treatment in a Small Animal Cancer Model

An appropriate small animal model is injected with a cancer cell type under test to induce tumor growth. After the tumors have grown, these test animals are extracorporeally treated with a porous biocompatible polymer sorbent and blood samples are taken at various time points to be analyzed for reduction in cytokines and chemokines, and in soluble or extracellular vesicle-associated checkpoint molecules. The tumors are also observed to detect a reduction in tumor growth. Animal body weight changes are compared to the non-treated animals.

For example, Wistar rats (n=6) are injected intravenously with 8×10⁷ splenocytes from leukemic donor rats. The percentage of CD19+CD5+ chronic lymphocytic leukemia (CLL) cells should be at least 80% of all viable lymphocytes. A 6-hour extracorporeal treatment with a porous biocompatible polymer sorbent is performed on days 1 through 6 post-injection. Rats in the control group (n=6) receive extracorporeal treatment with no sorbent device connected in the extracorporeal circuit. Blood is drawn on day 1 (0 h, 2 h, 4 h, 6 h, 24 h), followed by once daily from day 2 through the end of study. End of study will be at a time-period (e.g., after 30 days) that is determined to achieve a fully leukemic state meeting established criteria for CLL in control rats. At end of study, peripheral blood is drawn via cardiac puncture with EDTA as anti-coagulant. Spleen sections are embedded in paraffin and stained with hematoxylin and eosin (HE) and antibodies against CD5 and B220. Plasma for analysis is obtained and analyzed for cytokines (e.g., CCL2, IL-10, GM-CSF, CXCL2, CXCL5 and TNF-α) at all time points via multiplex assays. Plasma levels of soluble checkpoint molecules (e.g., sPD-L1 and sPD-L2) are measured by ELISA and extracellular vesicle-associated checkpoint molecules (e.g., EV-associated PD-L1) are also analyzed after collection of EVs using standard procedures.

### Example 5: Evaluating Removal of Soluble Checkpoint Inhibitors Protein Molecules from CytoSorb Using Bovine Whole Blood in a Small-Scale Benchtop Recirculation Study

Objective: To evaluate the efficiency of CytoSorb in removing eleven soluble ligands sPD-L1, sCTLA-4, sLAG-3, sTIM-3, sPD-1, sPD-L2, sICOS, and sVISTA from circulating bovine whole blood in a small-scale recirculation setup with 60 mL CytoSorb at a flow rate of 60 mL/min.

Experiment 1a: Blood was spiked with a mixture of checkpoint molecules sPD-L1, sCTLA-4, sLAG-3 and sTIM-3 at starting concentrations of 1400, 2000, 3000, and 4000 μg/mL respectively, and their removal by CytoSorb was assessed.

Experiment 1b: Blood was spiked with a mixture of checkpoint molecules sPD-1, sPD-L2, sICOS, and sVISTA at starting concentrations of 2000, 5000, 2000, and 2000 μg/mL respectively, and their removal by CytoSorb was assessed.

Rationale: The starting blood concentration for these studies is based on the clinically relevant maximum plasma concentrations found in the literature. [Zhao, J. et al. Plasma levels of soluble programmed death ligand-1 may be associated with overall survival in nonsmall cell lung cancer patients receiving thoracic radiotherapy. Medicine (Baltimore) 96, e6102 (2017); Zhou, J. et al. Soluble PD-L1 as a Biomarker in Malignant Melanoma Treated with Checkpoint Blockade. Cancer Immunol Res 5, 480-492 (2017); Botticelli, A. et al. The Role of Soluble LAG3 and Soluble Immune Checkpoints Profile in Advanced Head and Neck Cancer: A Pilot Study. J Pers Med 11 (2021); Keane, C. et al. LAG3: a novel immune checkpoint expressed by multiple lymphocyte subsets in diffuse large B-cell lymphoma. Blood Adv 4, 1367-1377 (2020); Shapiro, M. et al. Lymphocyte activation gene 3: a novel therapeutic target in chronic lymphocytic leukemia. Haematologica 102, 874-882 (2017); Odagiri, N. et al. Early Change in the Plasma Levels of Circulating Soluble Immune Checkpoint Proteins in Patients with Unresectable Hepatocellular Carcinoma Treated by Lenvatinib or Transcatheter Arterial Chemoembolization. Cancers (Basel) 12 (2020); Xie, C. et al. Soluble B7-H3 promotes the invasion and metastasis of pancreatic carcinoma cells through the TLR4/NF-kappaB pathway. Sci Rep 6, 27528 (2016); Noubissi Nzeteu, G. A. et al. Macrophage Differentiation and Polarization Regulate the Release of the Immune Checkpoint Protein V-Domain Ig Suppressor of T Cell Activation. Front Immunol 13, 837097 (2022).]

The selection of analytes (sPD-L1, sTIM-3, sLAG-3, sCTLA-4, sPD-1, sPD-L2, sICOS, sVISTA) was based on their co-existence in clinical samples in common cancer types. Notably, it has been established in a pre-clinical study that blocking LAG-3 alone did not restore T cell exhaustion, however, the combination of LAG-3/PD-1 blockade resulted in reduced tumor volume. [Hinshaw, et al. The Tumor Microenvironment Innately Modulates Cancer Progression. Cancer Res 79, 4557-4566 (2019).] This indicates that more broad spectrum removal of checkpoint molecules may prove to be of greater clinical significance than targeting a single checkpoint molecule. This is consistent with the fact that a number of clinical trials are underway to test bispecific antibodies as checkpoint inhibitors, for example a combination of LAG-3/PD-L1, LAG-3/TIM-3, or VISTA/PD-L1 to treat various cancer types where these combinations co-exist. [Lee, et al. Clinical Insights Into Novel Immune Checkpoint Inhibitors. Front Pharmacol 12, 681320 (2021).]

### Results and Discussion

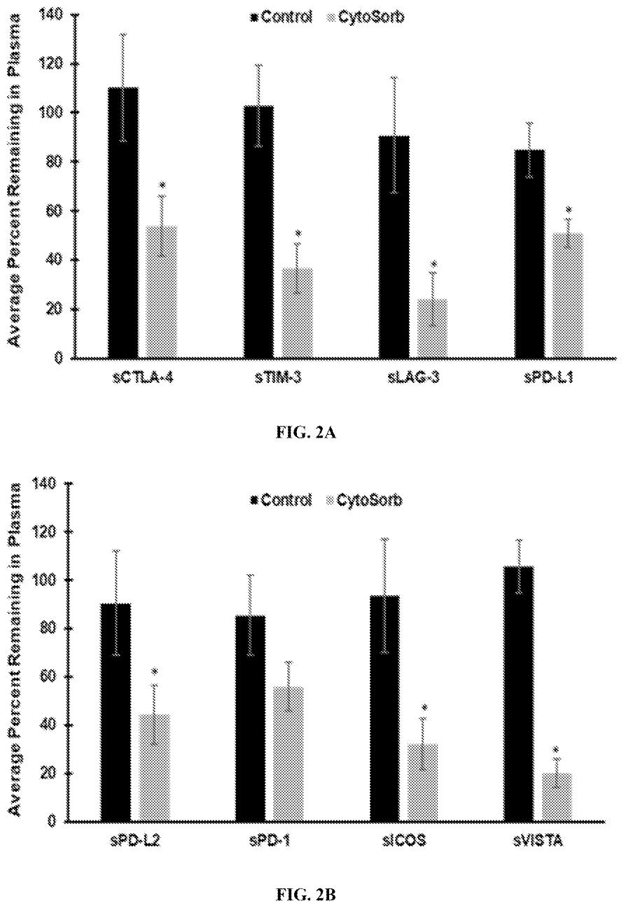

FIG. 2A shows the percent remaining of 4 proteins (sCTLA-4, sTIM-3, sLAG-3 and sPD-L1 during 6 h of hemoperfusion using CytoSorb in a benchtop recirculation small scale experiment. FIG. 2B shows the percent remaining of 4 proteins (sPD-1, sPD-L2, sVISTA and sICOS) during 6 h of hemoperfusion using CytoSorb in a benchtop recirculation small scale experiment.

The data is presented as a percent of baseline (0 h time point) for each protein for both control and treatment circuits as an average of three experiments. The control circuit results indicate that the tested proteins were stable over a period of 6 h where measured percent of baseline remained between 85-110% (FIGS. 2A, 2B). Therefore, a decrease in percent remaining of these proteins in the CytoSorb group can be solely accounted for their removal by the CytoSorb beads. The multiplex data indicates that the percent remaining in the CytoSorb group ranged from 20-56% at the end of 6 h and the difference between control and CytoSorb group was found to be significant (p<0.05) at 6 h for each protein (except for PD-1 where only two replicates were run, precluding statistical testing).

Specifically, average percent removal at the end of 6 h of hemoperfusion through CytoSorb was measured to be VISTA (80%), LAG-3 (76%), ICOS (68%), TIM-3 (63%), sPD-L2 (56%), sPD-L1 (49%), sCTLA-4 (46%) and sPD-1 (44%).

Removal by CytoSorb depends on various factors including 1) molecular weight 2) concentration and 3) molecular structure. In this experiment, we utilized a mixture of several checkpoint molecules to better mimic the clinical situation where several checkpoint molecules are in circulation simultaneously. Some of the molecules present simultaneously represent receptor-ligand, notably sPD-1 and PDL-2, where receptor-ligand binding during the experiment is expected. While, based on the factors above, we expect removal of each molecule when present individually, significant removal of both sPD-1 and sPD-L2 were seen despite their simultaneous presence, and with the ligand in excess to promote binding to its cognate receptor. Despite this, robust removal of sPD-1 was still achieved. Removal can be enhanced by increasing the dose of CytoSorb and/or prolonging treatment beyond 6 hours (see FIGS. 2A, 2B).

### Example 6: Use of Bioresorbable Porous Polymeric Materials for Intratumoral, Subcutaneous, Topical, Injectable and Oral Applications for the Treatment of Cancer by Adsorption of Soluble Immune Checkpoint Molecules and/or Cytokines

Bioresorbable porous polymeric materials that demonstrate a desired degradation profile with biocompatible and safe degradation products are used for the treatment of cancer by a local injection into the tumor, intravenous injection for blood cancers, oral application for gut and stomach cancers and topical and subcutaneous application for skin cancers such as melanoma.

One example is, poly(lactic-co-glycolic acid) [PLGA] based porous nano, macro or microparticles. These porous particles can be modified using various ligands or porogens to increase the adsorption of certain proteins. The degradation profile of these resorbable polymers can be varied from hours to months to years depending on the application type. Other bioresorbable polymers used are polyesters, polyanhydrides, polycaprolactone, polytrioxanes, polycarbonates and their combinations. These porous particles work by adsorbing soluble immune checkpoint protein molecules from the systemic circulation or local tumor environment and block the interactions between the soluble forms and membrane bound forms thereby preventing cancer cells to escape the immune response by activating T cells. These porous resorbable particles can be tested for treatment of various cancer types using methods described in Examples 3 and 4.

### Example 7: Use of the Sorbent Polymer for Removal of Excess Inflammatory Mediators and Checkpoint Molecules to Mitigate Inflammation-Driven Complications and Improve Effectiveness of Hyper/Hypothermia Cancer Treatments

Application of the sorbent prior to, during, and/or after treatment with hyper/hypothermia will result in removal of cytokines, chemokines, soluble checkpoint molecules, and other inflammatory factors that can interfere with efficacy or tolerability of the cancer treatment. For example, application of sorbent before hyper/hypothermia will remove anti-inflammatory cytokines such as IL-10 and TGF-β or soluble checkpoint molecules such as sPD-L1 that mediate a tumor-protective environment, resulting in improved tumor lysis and immune-mediated cancer cell apoptosis when hyper/hypothermia is applied. When the sorbent is applied during hyper/hypothermia, it will remove excess pro-inflammatory factors including IL-6 and TNF-α induced by the therapy that would otherwise cause damage to healthy tissue and improve tolerability of the treatment. Sorbent applied after hyper/hypothermia can remove inflammatory factors and damage associated molecular patterns released as cancer cell apoptosis continues post treatment and prevent development of cytokine release syndrome (CRS) or tumor lysis syndrome (TLR).

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

What is claimed:

1. A method of treating cancer in a subject, comprising treating a therapeutically effective amount of a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

2. A method of treating cancer in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer; and
wherein the administering places the sorbent in contact with a physiologic fluid of the subject.

3. The method of claim 1 or claim 2, wherein the sorbent is administered extracorporeally.

4. The method of claim 1 or claim 2, wherein the sorbent is administered intravenously, intramuscularly, intratumorally, intradermally, intraperitoneally, subcutaneously, topically, orally, nasally, via a feeding tube, or rectally.

5. A method of treating cancer in a subject in need thereof, comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

6. The method of claim 5, wherein the sorbent is administered intravenously, intramuscularly, intratumorally, intradermally, intraperitoneally, subcutaneously, topically, orally, nasally, via a feeding tube, or rectally.

7. The method of claim 5, wherein the sorbent is extracorporeal.

8. The method of any one of claims 2-7, wherein the physiologic fluid is blood.

9. A method of treating cancer in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

10. The method of any one of claims 1-9, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

11. The method of any one of claims 1-10, wherein the sorbent is produced using at least one crosslinking agent and at least one monomer.

12. The method of claim 11, wherein the monomer comprises one or more of divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, and divinylformamide.

13. The method of claim 11 or claim 12, wherein the crosslinking agent comprises one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

14. The method of any one of claims 11-13, wherein the sorbent is produced additionally utilizing at least one dispersing agent and at least one porogen.

15. The method of any one of claims 1-14, wherein the sorbent comprises a biocompatible and hemocompatible exterior coating that is covalently bound to the sorbent by free-radical grafting.

16. The method of any one of claims 1-15, wherein said sorbent is administered in combination with an anticancer agent, anticancer treatment, or an immunotherapeutic agent.

17. The method of claim 16, wherein said sorbent is administered prior to or at the same time as said anticancer agent, anticancer treatment, or immunotherapeutic agent.

18. The method of claim 16, wherein said sorbent is administered following administration of the anticancer agent, anticancer treatment, or immunotherapeutic agent.

19. The method of claim 17 or claim 18, wherein the anticancer agent, anticancer treatment, or immunotherapeutic agent comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

20. The method of any one of claims 1-19, wherein the sorbent reduces the concentration of one or more of cytokines, inflammatory mediators, toxins, electrolytes, chemicals, cellular debris, and metabolic waste products in the patient relative to an untreated subject.

21. The method of any one of claims 1-20, wherein the method reduces the entry of CAR-T cells and other activated cells into the central nervous system relative to an untreated subject.

22. The method of any one of claims 1-20, wherein the method reduces inhibition of the immune system.

23. The method of any one of claims 1-20, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

24. The method of any one of claims 1-23, wherein administration of the sorbent reduces inhibitors of the immune system before, during or after administration of the anticancer agent, anticancer treatment, or immunotherapeutic agent.

25. The method of any one of claims 1-23, wherein administration of the sorbent results in removal of one or more of a) cytokine or chemokine, b) soluble checkpoint molecule, and c) extracellular vesicle associated with a checkpoint molecule from bodily fluid of the subject.

26. The method of claim 25, wherein the soluble or extracellular vesicle-associated checkpoint molecule is one or more of the examples found in Table I.

27. The method of claim 25, wherein the cytokine or chemokine comprises one or more of a member of the interleukin, interferon, tumor growth factor, or tumor necrosis factor family.

28. The method of any one of claims 1-27, wherein the sorbent comprises one or more residues of divinylbenzene and ethylvinylbenzene, styrene, and ethylstyrene monomers.

29. A method of reducing inflammation related complications of (a) cancer or (b) cancer therapy or immunotherapy in a subject comprising treating the subject with a therapeutically effective amount of a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

30. A method of reducing inflammation related complications of (a) cancer or (b) cancer therapy or immunotherapy in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer; and
wherein the administering places the sorbent in contact with a physiologic fluid of the subject.

31. The method of claim 29 or claim 30, wherein the sorbent is administered extracorporeally.

32. The method of claim 29 or claim 30, wherein the sorbent is administered intravenously, intramuscularly, intratumorally, intradermally, intraperitoneally, subcutaneously, topically, orally, nasally, via a feeding tube, or rectally.

33. A method of reducing inflammation related complications of (a) cancer or (b) cancer therapy or immunotherapy in a subject in need thereof comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

34. The method of claim 33, wherein the contacting comprises administering the sorbent intravenously, intramuscularly, intratumorally, intradermally, intraperitoneally, subcutaneously, topically, orally, nasally, via a feeding tube, or rectally.

35. The method of claim 33, wherein the sorbent is extracorporeal.

36. The method of any one of claims 30-35, wherein the physiologic fluid is blood.

37. A method of reducing inflammation related complications of (a) cancer or (b) cancer therapy or immunotherapy in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

38. The method of any one of claims 29-37, wherein the method reduces inflammation related complication of cancer.

39. The method of any one of claims 29-37, wherein the method reduces inflammation due to cancer therapy.

40. The method of any one of claims 29-37, wherein the method reduces inflammation due to immunotherapy.

41. The method of claim 39 or claim 40, wherein the cancer therapy or immunotherapy comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

42. The method of any one of claims 38-40, wherein reducing inflammation improves one or more of the manifestation, severity, or progression of cachexia.

43. The method of any one of claims 29-42, wherein the method reduces the entry of CAR-T cells and other activated cells into the central nervous system relative to an untreated subject.

44. The method of any one of claims 29-42, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

45. The method of any one of claims 29-42, wherein the sorbent reduces the concentration of one or more of cytokines, chemokines, inflammatory mediators, toxins, electrolytes, chemicals, cellular debris, and metabolic waste products in the patient relative to an untreated subject.

46. The method of any one of claims 29-45, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

47. The method of any one of claims 29-46, wherein the sorbent is produced using at least one crosslinking agent and at least one monomer.

48. The method of claim 47, wherein the monomer comprises divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

49. The method of claim 47 or claim 48, wherein the crosslinking agent comprises one or more or divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, and divinylformamide.

50. The method of any one of claims 47-49, wherein the sorbent is produced additionally utilizing at least one dispersing agent and at least one porogen.

51. The method of any one of claims 29-50, wherein the sorbent comprises a biocompatible and hemocompatible exterior coating that is covalently bound to the sorbent by free-radical grafting.

52. The method of any one of claims 29-51, wherein the sorbent comprises one or more residues of divinylbenzene and ethylvinylbenzene, styrene, and ethylstyrene monomers.

53. A method of improving immune system function in a subject before, during or after treatment with an anticancer agent, anticancer treatment, and/or immunotherapeutic agent, comprising administering to the subject a therapeutically effective amount of a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

54. A method of improving immune system function in a subject in need thereof before, during or after treatment with an anticancer agent, anticancer treatment, and/or immunotherapeutic agent comprising administering a porous biocompatible sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer; and
wherein the administering places the sorbent in contact with a physiologic fluid of the subject.

55. The method of claim 53 or claim 54, wherein the sorbent is administered extracorporeally.

56. The method of claim 53 or claim 54, wherein the sorbent is administered intravenously, intramuscularly, intratumorally, intradermally, intraperitoneally, subcutaneously, topically, orally, nasally, via a feeding tube, or rectally.

57. A method of improving immune system function in a subject in need thereof before, during or after treatment with an anticancer agent, anticancer treatment, or immunotherapeutic agent comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

58. The method of claim 57, wherein the contacting comprises administering the sorbent intravenously, intramuscularly, intratumorally, intradermally, intraperitoneally, subcutaneously, topically, orally, nasally, via a feeding tube, or rectally.

59. The method of claim 57, wherein the sorbent is extracorporeal.

60. The method of any one of claims 54-59, wherein the physiologic fluid is blood.

61. A method of improving immune system function in a subject in need thereof before, during or after treatment with an anticancer agent, anticancer treatment, or immunotherapeutic agent comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

62. The method of any one of claims 53-61, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

63. The method of any one of claims 53-62, wherein the sorbent is produced using at least one crosslinking agent and at least one monomer.

64. The method of claim 63, wherein the monomer comprising divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

65. The method of claim 63 or claim 64, wherein the crosslinking agent comprising one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, or divinylformamide.

66. The method of any one of claims 63-65, wherein the sorbent is produced additionally utilizing at least one dispersing agent and at least one porogen.

67. The method of any one of claims 53-66, wherein the sorbent comprises a biocompatible and hemocompatible exterior coating that is covalently bound to the sorbent by free-radical grafting.

68. The method of any one of claims 53-67, wherein the sorbent comprises one or more residues of divinylbenzene and ethylvinylbenzene, styrene, and ethylstyrene monomers.

69. The method of any one of claims 53-68, wherein the anticancer agent, anticancer treatment, or immunotherapeutic agent comprises one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

70. The method of any one of claims 53-69, wherein the method improves immune system function in a subject in need thereof before treatment with an anticancer agent, anticancer treatment, or immunotherapeutic agent.

71. The method of any one of claims 53-69, wherein the method improves immune system function in a subject in need thereof during treatment with an anticancer agent, anticancer treatment, or immunotherapeutic agent.

72. The method of any one of claims 53-69, wherein the method improves immune system function in a subject in need thereof after treatment with an anticancer agent, anticancer treatment, or immunotherapeutic agent.

73. The method of one or more of claims 70-72, wherein administration of the sorbent results in removal of one or more of a) cytokine or chemokine, b) soluble checkpoint molecule, and c) extracellular vesicle associated with a checkpoint molecule from bodily fluid of the subject.

74. The method of claim 73, wherein the soluble or extracellular vesicle-associated checkpoint molecule is one or more of the examples found in Table I.

75. The method of claim 73, wherein the cytokine or chemokine comprises one or more of a member of the interleukin, interferon, tumor growth factor, or tumor necrosis factor family.

76. The method of any one of claims 53, 54, 57, or 61, wherein the improved immune system function includes a reduction in systemic or local immune suppression.

77. The method of any one of claims 53, 54, 57, or 61, wherein the improved immune system function includes a reduction in systemic or local hyperinflammation.

78. A method of improving effectiveness of cancer therapy or immunotherapy in a subject, comprising treating the subject with a therapeutically effective amount of a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

79. A method of improving effectiveness of cancer therapy or immunotherapy in a subject in need thereof comprising administering a porous biocompatible polymer sorbent to the subject, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer; and
wherein the administering places the sorbent in contact with a physiologic fluid of the subject.

80. The method of claim 78 or claim 79, wherein the sorbent is administered extracorporeally.

81. The method of claim 78 or claim 79, wherein the sorbent is administered intravenously, intramuscularly, intratumorally, intradermally, intraperitoneally, subcutaneously, topically, orally, nasally, via a feeding tube, or rectally.

82. A method of improving effectiveness of cancer therapy or immunotherapy in a subject in need thereof comprising contacting a physiologic fluid of the subject with a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

83. The method of claim 82, wherein the contacting comprises administering the sorbent intravenously, intramuscularly, intratumorally, intradermally, intraperitoneally, subcutaneously, topically, orally, nasally, via a feeding tube, or rectally.

84. The method of claim 82 wherein the sorbent is extracorporeal.

85. The method of any one of claims 79-84, wherein the physiologic fluid is blood.

86. A method of improving effectiveness of cancer therapy or immunotherapy in a subject in need thereof comprising providing to the subject a porous biocompatible polymer sorbent, wherein
the sorbent comprises a range of pore diameters between about 50 Å to about 3000 Å and a pore volume between about 0.5 cc/g to about 3.0 cc/g dry polymer; or
the sorbent comprises a range of pore diameters between about 50 Å to about 10,000 Å and a pore volume between about 0.5 cc/g to about 4.0 cc/g dry polymer; or
the sorbent comprises a range of pore diameters between about 50 Å to about 40,000 Å and a pore volume between about 0.5 cc/g to about 5.0 cc/g dry polymer.

87. The method of any one of claims 78-86, wherein the sorbent comprises a pore structure such that the total pore volume of pore size in the range of 50 Å to 40,000 Å is greater than 0.5 cc/g to 5.0 cc/g dry sorbent; wherein the ratio of pore volume between 50 Å to 40,000 Å (pore diameter) to pore volume between 1,000 Å to 10,000 Å (pore diameter) of the sorbent is smaller than 2:1.

88. The method of any one of claims 78-87, wherein the sorbent is produced using at least one crosslinking agent and at least one monomer.

89. The method of claim 88, wherein the monomer comprising divinylbenzene and ethylvinylbenzene, styrene, ethylstyrene, acrylonitrile, butyl methacrylate, octyl methacrylate, butyl acrylate, octyl acrylate, cetyl methacrylate, cetyl acrylate, ethyl methacrylate, ethyl acrylate, vinyltoluene, vinylnaphthalene, vinylbenzyl alcohol, vinylformamide, methyl methacrylate, methyl acrylate, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythritol dimethacrylate, pentaerythritol trimethacrylate, pentaerythritol tetramethacrylate, pentaerythritol diacrylate, pentaerythritol triacrylate, pentaerythritol tetraacrylate, dipentaerythritol dimethacrylate, dipentaerythritol trimethacrylate, dipentaerythritol tetramethacrylate, dipentaerythritol diacrylate, dipentaerythritol triacrylate, dipentaerythritol tetraacrylate, divinylformamide and mixtures thereof.

90. The method of claim 88 or claim 89, wherein the crosslinking agent comprising one or more of divinylbenzene, trivinylbenzene, divinylnaphthalene, trivinylcyclohexane, divinylsulfone, trimethylolpropane trimethacrylate, trimethylolpropane dimethacrylate, trimethylolpropane triacrylate, trimethylolpropane diacrylate, pentaerythrital dimethacrylates, pentaerythrital trimethacrylates, pentaerythrital, tetramethacrylates, pentaerythritol diacrylates, pentaerythritol triacrylates, pentaerythritol tetraacrylates, dipentaerythritol dimethacrylates, dipentaerythritol trimethacrylates, dipentaerythritol tetramethacrylates, dipentaerythritol diacrylates, dipentaerythritol triacrylates, dipentaerythritol tetraacrylates, or divinylformamide.

91. The method of any one of claims 88-90, wherein the sorbent is produced additionally utilizing at least one dispersing agent and at least one porogen.

92. The method of any one of claims 78-91, wherein the sorbent comprises a biocompatible and hemocompatible exterior coating that is covalently bound to the sorbent by free-radical grafting.

93. The method of any one of claims 78-92, wherein the sorbent comprises one or more residues of divinylbenzene and ethylvinylbenzene, styrene, and ethylstyrene monomers.

94. The method of any one of claims 78-93, wherein the cancer therapy or immunotherapy comprises administration of one or more of radiotherapy, chemotherapy, hypothermia, hyperthermia, checkpoint inhibitors, monoclonal antibodies, bi-specific T-cell engagers, CAR-T cell immunotherapies, cancer vaccines, and oncolytic viruses.

95. The method of any one of claims 78-94, wherein the method improves the effectiveness of cancer therapy.

96. The method of any one of claims 78-94, wherein the method improves the effectiveness of immunotherapy.

97. The method of any one of claims 78-96, wherein the sorbent reduces the concentration of one or more of cytokines, chemokines, inflammatory mediators, toxins, electrolytes, chemicals, cellular debris, and metabolic waste products in the patient relative to an untreated subject.

98. The method of any one of claims 78-96, wherein the method reduces the entry of CAR-T cells and other activated cells into the central nervous system relative to an untreated subject.

99. The method of any one of claims 78-96, wherein the method reduces the damage by one or more of cytokine release syndrome (CRS), CAR-T-cell-related encephalopathy syndrome (CRES), Chimeric Antigen Receptor T Cell Therapy-Associated Toxicity (CARTOX), Immune Effector Cell-Associated Neurotoxicity (ICANS), or tumor lysis syndrome (TLS) relative to an untreated subject.

100. The method of any one of claims 78-96, wherein the method improves one or more of the manifestation, severity, or progression of cachexia.

101. The method of one or more of claims 78-96, wherein administration of the sorbent results in removal of one or more of a) cytokine or chemokine, b) soluble checkpoint molecule, and c) extracellular vesicle associated with a checkpoint molecule from bodily fluid of the subject.

102. The method of claim 101, wherein the soluble or extracellular vesicle-associated checkpoint molecule is one or more of the examples found in Table I.

103. The method of claim 101, wherein the cytokine or chemokine comprises one or more of a member of the interleukin, interferon, tumor growth factor, or tumor necrosis factor family.
